SEQUENCE LISTING (18 sequences)

SEQ ID NO: 1 - protein, 20 aa, Artificial Sequence
  Leader Peptide
MSRSVALAVLALLSLSGLEA

SEQ ID NO: 2 - protein, 9 aa, Artificial Sequence
  Peptide antigen STEAP1
MLAVFLPIV

SEQ ID NO: 3 - protein, 8 aa, Artificial Sequence
  Peptide antigen Ova
SIINFEKL

SEQ ID NO: 4 - protein, 15 aa, Artificial Sequence
  Linker1 (disulfide trap stabilized)
GGGGSGGGGSGGGGS

SEQ ID NO: 5 - protein, 15 aa, Artificial Sequence
  Linker1 (disulfide trap stabilized)
GCGASGGGGSGGGGS

SEQ ID NO: 6 - protein, 99 aa, Artificial Sequence [sequence omitted]

SEQ ID NO: 7 - protein, 20 aa, Artificial Sequence
  Linker2
GGGGSGGGGSGGGGSGGGGS

SEQ ID NO: 8 - protein, 182 aa, Artificial Sequence
  [Alpha] 1 & 2 domain
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRA

SEQ ID NO: 9 - protein, 182 aa, Artificial Sequence
  Murine H2Kb [alpha]1 & 2 domain (Y84C)
GPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRT

SEQ ID NO: 10 - protein, 182 aa, Artificial Sequence [sequence omitted]

SEQ ID NO: 11 - protein, 113 aa, Artificial Sequence
  sequence of HLA-G [alpha]3
DPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 12 - protein, 11 aa, Artificial Sequence
  Factor Xa restriction site
IEGRTGTKLGP

SEQ ID NO: 13 - protein, 10 aa, Artificial Sequence
  Myc tag
EQKLISEEDL

SEQ ID NO: 14 - protein, 6 aa, Artificial Sequence
  His tag
HHHHHH

SEQ ID NO: 15 - protein, 469 aa, Artificial Sequence
  dtH2Gova
SIINFEKLGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLIEGRTGTKLGPEQKLISEEDLNSAVDHHHHHH

SEQ ID NO: 16 - protein, 470 aa, Artificial Sequence
  dtGsteap
MLAVFLPIVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLIEGRTGTKLGPEQKLISEEDLNSAVDHHHHHH

SEQ ID NO: 17 - protein, 650 aa, Artificial Sequence
  ILT2
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 18 - protein, 598 aa, Artificial Sequence
  ILT4
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

(19) United States
(12) Patent Application Publication
(10) Pub. No.: US 2020/0157175 A1
(43) Pub. Date: May 21, 2020

(54) COMBINATIONS OF MHC CLASS IB MOLECULES AND PEPTIDES FOR TARGETED THERAPEUTIC IMMUNOMODULATION

(71) Applicants: Valentin BRUTTEL, (US); JULIUS-MAXIMILIANS-UNIVERSITÄT WÜRZBURG, Würzburg (DE)

(72) Inventors: Valentin BRUTTEL, Würzburg (DE); Jörg WISCHHUSEN, Würzburg (DE)

(21) Appl. No.: 16/615,188

(22) PCT Filed: May 18, 2018
(86) PCT No.: PCT/EP2018/063100

§ 371 Date: Nov. 20, 2019

(30) Foreign Application Priority Data
May 23, 2017 (EP) 17172444.6

(51) Int. Cl. C07K 14/74; A61K 39/00; A61K 39/395; A61K 38/17

(52) U.S. Cl. CPC C07K 14/70539; A61K 39/001189; A61K 2039/605; A61K 38/1774; A61K 39/3955

(57) ABSTRACT

The present invention relates to therapeutical uses of non-classical major histocompatibility complex (MHC), also known as MHC class lb molecules in combination with defined peptides. The invention more specifically relates to targeted immunomodulatory effects of defined peptides in combination with proteins comprising one or more domains of a non-classical MHC class lb molecule or in combination with molecules that interfere with the interaction of MHC class lb molecules and their receptors. The invention also relates to methods of producing such proteins, pharmaceutical compositions comprising the same, as well as their uses for treating medical conditions in which antigen-specific immune reactions are beneficial, including cancer and infectious diseases, or harmful, including autoimmune diseases, organ/tissue rejection, immune reactions towards pharmaceutical compounds or reproductive disorders. Moreover, as the invention reveals a novel mode of action for MHC class lb molecules during antigen-specific tolerance induction, it also relates to methods for interfering with this mechanism in situation where induction of antigen-specific immune tolerance is wanted, but physiologically prevented by said mechanism.

### FIELD OF THE INVENTION

The present invention relates to therapeutical uses of non-classical human major histocompatibility complex (MHC) molecules (also named MHC class Ib molecules) in combination with peptide antigens. The invention more specifically relates to peptide antigens in combination with proteins comprising one or more domains of a non-classical MHC class Ib molecule or in combination with molecules that inhibit binding of MHC class Ib molecules to their receptors. The invention also relates to methods of producing such proteins, pharmaceutical compositions comprising the same, as well as their uses for treating medical conditions in which antigen-specific immune reactions are beneficial, including cancer and infectious diseases, or harmful, including autoimmune diseases, organ/tissue rejection, immune reactions towards pharmaceutical compounds or reproductive disorders.

### BACKGROUND

Three main classes of Major histocompatibility complex (MHC) antigens are known, namely class I antigens (HLA-A, B, C, E, F, G), class II antigens (HLA-DP, HLA-DQ and HLA-DR) and class III antigens. Class I antigens include conventional/classical MHC Ia antigens, HLA-A, HLA-B and HLA-C, as well as non-classical MHC Ib antigens HLA-E, HLA-F, and HLA-G. Class I antigens comprise 3 globular domains ([alpha]1, [alpha]2 and [alpha]3). MHC I complexes further comprise a beta-2-microglobulin and a presented peptide that is bound in a peptide binding cleft comprising the [alpha]1 and [alpha]2 domains. Thus, peptide-loaded conventional MHC Ia molecules can initiate peptide-specific, T cell mediated immune responses which may lead to lysis of the presenting cell. This mechanism is vital for vaccination strategies that may include shorter or longer peptides (Slingluff, Cancer J. 2011 September; 17(5): 343-350), nucleic acids coding for antigens (Restifo et al., Gene Ther. 2000 January; 7(2): 89-92), proteins or often attenuated organisms are developed or clinically used to induce immune reactions towards specific antigens. Antigens may include viral, bacterial or tumor associated antigens.

Unlike conventional MHC Ia molecules, which are expressed in most human tissues, non-classical MHC Ib antigens such as HLA-G show only very restricted tissue expression. Physiologically, high levels of HLA-G are expressed by extravillous trophoblasts of the normal human placenta, where they likely function as immunomodulatory agents protecting the foetus from the maternal immune system (absence of rejection by the mother). In line with this hypothesis previous studies have shown that HLA-G proteins are able to inhibit allogeneic responses such as proliferative T lymphocyte cell response, cytotoxic T lymphocytes mediated cytolysis, and NK cells mediated cytolysis (Rouas-Freiss N. et al., Proc. Natl. Acad. Sci., 1997, 94, 5249-5254; Semin Cancer Biol 1999, vol 9, p. 3).

The sequence of the HLA-G gene has been described (e.g., Geraghty et al. Proc. Natl. Acad. Sci. USA, 1987, 84, 9145-9149; Ellis; et al., J. Immunol., 1990, 144, 731-735) and comprises 4396 base pairs. This gene is composed of 8 exons, 7 introns and a 3′ untranslated end, corresponding respectively to the following domains: exon 1: signal sequence, exon 2: [alpha]1 extracellular domain, exon 3: [alpha]2 extracellular domain, exon 4: [alpha]3 extracellular domain, exon 5: transmembrane region, exon 6: cytoplasmic domain I, exon 7: cytoplasmic domain II (untranslated), exon 8: cytoplasmic domain III (untranslated) and 3′ untranslated region. Seven isoforms of HLA-G have been identified, among which 4 are membrane bound (HLA-G1, HLA-G2, HLA-G3 and HLA-G4) and 3 are soluble (HLA-G5, HLA-G6 and HLA-G7) (see e.g., Carosella et al., Blood 2008, vol. 111, p 4862). The mature HLA-G1 protein isoform comprises the three external domains (α1-α3), the transmembrane region and the cytoplasmic domain, the mature HLA-G5 protein isoform comprises the three external domains (α1-α3) and a short sequence coded by intron 4, but lacks transmembrane and intracellular domains. All soluble HLA-G isoforms lack the transmembrane and cytoplasmic domains and may also be produced by cleavage of membrane bound isoforms.

HLA-G interacts in a peptide-independent manner with specific receptors such as Kir2DL4, ILT2 (LILRB1) and ILT4 (LILRB2, Clements et al., Proc Natl Acad Sci USA. 2005 Mar. 1; 102(9):3360-5) The most prominent immunosuppressive effects of HLA-G on T cells are mediated by ILT2 and ILT4. As these receptors interact with the [alpha]-3 domain contained in HLA-G but also in other MHC class Ib molecules such as HLA-F (Lepin et al., Eur. J. Immunol. 2000. 30: 3552-3561), [alpha]-3 domain-dependent effects observed for the representative MHC class Ib molecule HLA-G can also be induced by alternative MHC class Ib molecules.

It is further known that MHC class Ib molecules present peptides via their the [alpha]1 and [alpha]2 domains. These peptides typically consist of 8-10 amino acids and contain certain anchor residues (Diehl et al. Curr Biol. 1996 Mar. 1; 6(3):305-14, Lee et al. Immunity. 1995 November; 3(5):591-600.). However, to the inventors' knowledge, peptide-specific interactions of human MHC class Ib molecules with cognate T cell receptors have not yet been investigated. Likewise, there are no clear data from animal models. While Swanson et al. suggested that murine MHC Ib molecules may induce peptide-specific immune responses (Swanson et al., An MHC class Ib-restricted CD8 T cell response confers antiviral immunity, JEM 2008), Wang et al. described suppression of peptide-specific immune responses by murine Qa2 molecules. (Wang et al., Sci. Rep. 36064, 31. Oct. 2016). However, human and murine MHC Ib molecules are very different (Pratheek et al., Indian J Hum Genet. 2014 April-June; 20(2): 129-141) As HLA-G and Qa-2 share only 67% of sequency identity as analyzed using proteinblast on the UniProtKB reference sequences Q5RJ85 (Q5RJ85_HUMAN) and P79568 (P79568_MOUSE), conclusions drawn from Qa-2 must be treated with great caution, and it cannot be predicted whether or not they also apply to human HLA-G. The considerable difficulties in defining mouse models which are suitable for studies of HLA-G function in basic science and preclinical research have recently been outlined in a review article (Nguyen-Lefebvre et al., 2016).

Based on the already available data, it has been proposed that HLA-G proteins may be used for treating graft rejection in allogeneic or xenogenic organ/tissue transplantation. HLA-G proteins have also been proposed for the treatment of hematological malignancies (EP1 054 688), inflammatory disorders (EP1 189 627) and, more generally, immune related diseases. Furthermore, HLA-G is frequently expressed by human tumors (Carosella et al. Trends Immunol. 2008 March; 29(3):125-32), where it is thought to function like a immunosuppressive immune checkpoint molecule that unspecifically suppresses immune responses in the tumor microenvironment (Carosella E D et al., Adv Immunol. 2015; 127:33-144). However, none of these studies analyzed the peptides presented on HLA-G. Consequently, the question of whether the presented peptides could direct the observed MHC class Ib mediated effects was not even raised.

Given the limitations inherent in all mouse models to study human MHC class Ib molecules, effects of such molecules on human T cells have to be explored in vitro in order to achieve a mechanistic understanding which then allows to predict MHC class Ib-dependent functions in vivo. In the context of antigen-specific immune responses, modulation of cytotoxic and tolerogenic T cells is critical. While cytotoxic CD8⁺ effector T cells (cytotoxic T lymphocytes, CTLs) and regulatory T cells (Treg) are both capable of detecting antigenic peptides presented on MHC molecules, CTL are capable of destroying cells expressing their cognate antigens whereas regulatory T cells are tissue-protective in particular when their cognate antigen is presented by the respective tissue (Wright et al., 2009 PNAS vol. 106 no. 45, 19078-83). Importantly, antigen-specific regulatory T cells can also exert a bystander effect and suppress immune responses towards other antigens if they are activated by their cognate antigen in the target tissue. CTL can thus be beneficial for cancer patients (Gajewski et al., Nat. Immunol. 14, 1014-1022, 2013) but harmful in autoimmune diseases. Treg cells which suppress immune responses play an opposing role. Insufficient activity or functionality of Treg results in severe autoimmune disease in mice and may also be linked to human autoimmune diseases (Bluestone et al., J Clin Invest. 2015; 125(6):2250-2260). Strategies for the inhibition (or de-inhibition) of cytotoxic T cells and for the induction (or inhibition) of Treg are therefore needed.

In the current clinical practice, diseases caused by pathological immune responses (e.g. autoimmune diseases) are usually treated with therapeutics that suppress immune responses irrespective of the targeted antigen, which can cause severe and often dose limiting side-effects and increase the risk for opportunistic infections. Thus, improved means and uses for the treatment of such diseases are needed. Consequently, there is a need in the art for improved means and uses for therapeutic modulation of the immune system by more targeted and antigen-specific means.

Conversely, there is also a need for improved means and uses for the treatment of diseases in which immune responses directed against specific antigens are desired, including cancers. For example, many of the vaccination approaches which have been described for cancer immunotherapy have been shown to be ineffective because of immunosuppression mechanisms exerted by the cancers. Therefore, improved means and uses for the treatment of such diseases including cancers are also needed.

### DESCRIPTION OF THE INVENTION

The inventors have surprisingly found that human MHC class Ib molecules such as HLA-G possess the ability to induce antigen-specific tolerance towards presented peptide antigens. Thus, albeit being of similar structure and sequence as classical human MHC class Ia molecules which induce antigen peptide-specific immune responses, MHC class Ib molecules can advantageously be used according to the invention to suppress immune responses in an antigen-specific manner. Antigen-specific suppression of immune responses towards defined antigens can be induced by eliminating antigen-specific cytotoxic T cells or by inducing antigen-specific regulatory T cells which recognize either the respective autoantigen or another target antigen expressed in the tissue prone to autoimmune attack. In accordance with the above the inventors have shown that both cytotoxic effector T cells can be eliminated (as exemplified in FIG. 1) and tolerogenic regulatory T cells can be induced (as exemplified in FIG. 7) in an antigen peptide-specific manner using an exemplary human MHC class Ib molecule. In non-limiting embodiments, these effects can be achieved by combining specific peptide antigens with either membrane-bound or soluble MHC class Ib molecules. Conversely, in situations where induction of antigen-specific immune responses is desired, MHC class Ib associated immune tolerance needs to be broken. The inventors have found that this can be achieved through agents that block the binding of human MHC Ib molecules to their receptors.

According to the invention, peptides in combination with MHC class Ib molecules can thus advantageously be used to suppress immune responses in an antigen-specific or tissue-specific manner. This represents a significant advantage as compared to many conventional therapeutics which suppress immune responses irrespective of the targeted antigen, as their lack of specificity causes severe and dose-limiting side-effects and increases the risk for opportunistic infections.

Additionally, the inventors have surprisingly found that for the suppression of immune responses according to the invention, molecules other than naturally occurring MHC class Ib molecules, and in particular polypeptides which only comprise at least one domain of an MHC class Ib molecule, preferably at least an [alpha]3 domain of an MHC class Ib molecule, can be used: As exemplified in FIGS. 7 and 8, the [alpha]1 and [alpha]2 domains of variable class I a molecules can be productively combined with the [alpha]3 domain of a human MHC class Ib molecule in order to suppress immune responses towards peptides presented by these antigens.

Thus, according to the invention, the use of, for example, an immunosuppressive [alpha]3 domain of an MHC class Ib molecule in combination with, for example, a targeting antigen presented by an MHC class I [alpha] 1 & 2 domain will be beneficial in many autoimmune diseases.

Conversely, the new and surprising findings of the inventors also indicate that the suppression of antigen-specific immune responses caused by MHC class Ib molecules can be reverted by agents that interfere with binding of MHC class Ib molecules to their receptors. Thus, according to the invention, such blocking agents such as antibodies to the MHC class Ib molecules (as exemplified in FIG. 2) or their receptors including ILT2 and ILT4 (as exemplified in FIG. 1) can advantageously be used for the treatment of diseases in which immune responses directed against specific antigens are desired. These include cancers such as gastric, gastro-intestinal stromal, head and neck, kidney, liver, lung, breast, uterine, ovarian, cervical, vulvar, vaginal, urothelial, testis, colon and intestinal, pancreatic, skin cancer and sarcoma (see, for instance, http://medicalgenome.kribb.re.kr/GENT/search/view_result.php), and infectious diseases, including but not limited to trypanosomiasis (see, for instance, Gineau et al., Clin Infect Dis. 2016 Nov. 1; 63(9):1189-1197), cytomegalovirus infection (see, for instance, Cosman et al., Immunity. 1997 August; 7(2):273-82), HTLV-1 infection (see, for instance, Cilião Alves et al., J Gen Virol. 2016 October; 97(10):2742-2752.), hepatitis C infection (see, for instance, Ding et al., Med Sci Monit. 2016 Apr. 26; 22:1398-402.) or malaria falciparum infection (see, for instance, Garcia et al., Infect Genet Evol. 2013 June; 16:263-9),

In situations where specific immune responses against selected antigens first need to be induced, vaccines comprising peptides or proteins or attenuated pathogens or protein-coding DNA or RNA are typically being used in the art. However, such vaccinations may fail to elicit a response or even induce unwanted tolerance (Slingluff, Cancer J. 2011 September; 17(5): 343-350). As tumor cells (Carosella et al. Trends Immunol. 2008 March; 29(3):125-32) and virally infected cells (Rizzo et al, Front Immunol. 2014; 5: 592) express MHC class Ib molecules such as HLA-G antigen presentation on MHC class Ib molecules may be responsible for such failures. Thus, according to the invention, agents that specifically block the binding of MHC class Ib molecules to their receptors can be used to increase the efficacy of therapies in which specific antigenic proteins or peptides are used to induce peptide-specific or protein-specific immune responses. These include therapies based on externally given vaccines, but can also be extended to therapies during which antigenic material released from dying tumor cells can induce antigen-specific T cell responses, such as radiotherapy or chemotherapy (see, for instance, Zitvogel et al., Nature Reviews Immunology 8, 59-73, January 2008, for such therapies). Conversely, unwanted vaccination effects as elicited by treatment with biologicals or by gene therapy may be counteracted by addition of MHC class Ib based constructs in order to prevent the occurrence of anti-drug antibodies.

Accordingly, the invention relates to the following preferred embodiments:
1. A pharmaceutical composition comprising:
   a) a human MHC class Ib molecule, or a polypeptide capable of presenting peptide antigens to T cells, wherein the polypeptide comprises an [alpha] 3 domain of a human MHC class Ib molecule or a derivative of an [alpha] 3 domain of a human MHC class Ib molecule, said derivative being capable of binding to ILT2 or ILT4, and
   b) a peptide antigen which is presented by said MHC class Ib molecule or polypeptide according to a).
2. The pharmaceutical composition according to item 1, wherein the composition comprises the polypeptide capable of presenting peptide antigens according to a), and wherein said polypeptide comprises, preferably in an N- to C-terminal order, an [alpha]1 and an [alpha]2 domain of an MHC class Ia molecule that is followed by said [alpha]3 domain or said derivative.
3. The pharmaceutical composition according to items 1 or 2, wherein the [alpha]3 domain or derivative comprised by said MHC class Ib molecule or polypeptide is identical to or has at least 80% amino acid sequence identity, preferably at least 90% amino acid sequence identity, with the [alpha]3 domain amino acid sequence of SEQ ID No: 11.
4. The pharmaceutical composition according to item 3, wherein the [alpha]3 domain or derivative comprised by said MHC class Ib molecule or polypeptide is identical to or has at least 92% amino acid sequence identity with the [alpha]3 domain amino acid sequence of SEQ ID No: 11.
5. The pharmaceutical composition according to item 3, wherein the [alpha]3 domain or derivative comprised by said MHC class Ib molecule or polypeptide is identical to or has at least 94% amino acid sequence identity with the [alpha]3 domain amino acid sequence of SEQ ID No: 11.
6. The pharmaceutical composition according to item 3, wherein the [alpha]3 domain or derivative comprised by said MHC class Ib molecule or polypeptide is identical to or has at least 96% amino acid sequence identity with the [alpha]3 domain amino acid sequence of SEQ ID No: 11.
7. The pharmaceutical composition according to item 3, wherein the [alpha]3 domain or derivative comprised by said MHC class Ib molecule or polypeptide is identical to or has at least 98% amino acid sequence identity with the [alpha]3 domain amino acid sequence of SEQ ID No: 11.
8. The pharmaceutical composition according to item 3, wherein the [alpha]3 domain or derivative comprised by said MHC class Ib molecule or polypeptide is identical to or has at least 99% amino acid sequence identity with the [alpha]3 domain amino acid sequence of SEQ ID No: 11.
9. The pharmaceutical composition according to item 3, wherein the [alpha]3 domain or derivative comprised by said MHC class Ib molecule or polypeptide is identical to the [alpha]3 domain amino acid sequence of SEQ ID No: 11.
10. The pharmaceutical composition according to any of the preceding items, wherein said MHC class Ib molecule according to a) or said polypeptide capable of presenting peptide antigens according to a) is capable of binding to ILT2 or ILT4 with an affinity constant K_{d} of less than 40 μM as measured by surface plasmon resonance spectroscopy.
11. The pharmaceutical composition according to any of the preceding items, wherein said MHC class Ib molecule according to a) or said polypeptide capable of presenting peptide antigens according to a) is capable of binding to ILT2 or ILT4 with an affinity constant K_{d} of less than 20 μM as measured by surface plasmon resonance spectroscopy.
12. The pharmaceutical composition according to any of the preceding items, wherein said MHC class Ib molecule according to a) or said polypeptide capable of presenting peptide antigens according to a) is capable of binding to ILT2 or ILT4 with an affinity constant K_{d} of less than 10 μM as measured by surface plasmon resonance spectroscopy.
13. The pharmaceutical composition according to any of the preceding items, wherein said pharmaceutical composition further comprises a polypeptide domain comprising the amino acid sequence of SEQ ID No: 6, or a sequence at least 90% identical to the amino acid sequence of SEQ ID No: 6, preferably at least 95% identical to the amino acid sequence of SEQ ID No: 6, more preferably at least 98% identical to the amino acid sequence of SEQ ID No: 6, and wherein said polypeptide domain is preferably comprised by the polypeptide capable of presenting peptide antigens according to a).
14. The pharmaceutical composition according to any of the preceding items, wherein said MHC class Ib molecule according to a) or said polypeptide capable of presenting peptide antigens according to a) further comprises one or more linker sequences, preferably (GGGGS)n linker sequences.
15. The pharmaceutical composition according to any of the preceding items, wherein said MHC class Ib molecule according to a) or said polypeptide capable of presenting peptide antigens according to a) is a dimer or multimer.
16. The pharmaceutical composition according to any of the preceding items, wherein the peptide antigen is 7 to 11 amino acids in length, preferably 8-10 amino acids in length.
17. The pharmaceutical composition according to any of items 1 and 3-16, wherein the composition comprises the MHC class Ib molecule according to a), and wherein the MHC class Ib molecule is HLA-E, HLA-F or HLA-G.
18. The pharmaceutical composition according to item 17, wherein the MHC class Ib molecule is HLA-G.
19. The pharmaceutical composition according to item 17 or 18, wherein the MHC class Ib molecule is a human MHC class Ib molecule.
20. The pharmaceutical composition according to any of the preceding items, wherein the peptide antigen according to b) is covalently bound to the MHC class Ib molecule or polypeptide according to a).
21. The pharmaceutical composition according to item 20, wherein the peptide antigen according to b) and the MHC class Ib molecule or polypeptide according to a) are covalently bound through a peptide bond and are part of a single polypeptide chain.
22. A recombinant polypeptide capable of presenting a peptide antigen, the recombinant polypeptide comprising, in an N- to C-terminal order,
   i) a peptide antigen presented by said recombinant polypeptide;
   ii) optionally a first linker sequence;
   iii) optionally a sequence of a human polypeptide domain comprising a sequence of a human β2 microglobulin, or an amino acid sequence at least 90% identical to the amino acid sequence of human β2 microglobulin represented by SEQ ID No: 6;
   iv) optionally a second linker sequence;
   v) optionally an [alpha] 1 domain of an MHC molecule;
   vi) optionally an [alpha] 2 domain of an MHC molecule;
   vii) an [alpha] 3 domain of an MHC Ib molecule or a derivative of an [alpha] 3 domain of an MHC class Ib molecule, said derivative being capable of binding to ILT2 or ILT4;
   viii) optionally a protease cleavage site; and
   ix) optionally an affinity tag.
23. The recombinant polypeptide according to item 22, wherein
   v) said [alpha]1 domain and vi) said [alpha]2 domain are from an MHC class Ia molecule.
24. The recombinant polypeptide according to items 22 or 23, wherein the [alpha]3 domain or derivative is identical to or has at least 80% amino acid sequence identity, preferably at least 90% amino acid sequence identity, with the [alpha]3 domain amino acid sequence of SEQ ID No: 11.
25. The recombinant polypeptide according to item 24, wherein the [alpha]3 domain or derivative is identical to or has at least 92% amino acid sequence identity with the [alpha]3 domain amino acid sequence of SEQ ID No: 11.
26. The recombinant polypeptide according to item 24, wherein the [alpha]3 domain or derivative is identical to or has at least 94% amino acid sequence identity with the [alpha]3 domain amino acid sequence of SEQ ID No: 11.
27. The recombinant polypeptide according to item 24, wherein the [alpha]3 domain or derivative is identical to or has at least 96% amino acid sequence identity with the [alpha]3 domain amino acid sequence of SEQ ID No: 11.
28. The recombinant polypeptide according to item 24, wherein the [alpha]3 domain or derivative is identical to or has at least 98% amino acid sequence identity with the [alpha]3 domain amino acid sequence of SEQ ID No: 11.
29. The recombinant polypeptide according to item 24, wherein the [alpha]3 domain or derivative is identical to or has at least 99% amino acid sequence identity with the [alpha]3 domain amino acid sequence of SEQ ID No: 11.
30. The recombinant polypeptide according to item 24, wherein the [alpha]3 domain is identical to the [alpha]3 domain amino acid sequence of SEQ ID No: 11.
31. The recombinant polypeptide according to any of the preceding items, wherein said polypeptide is capable of binding to ILT2 or ILT4 with an affinity constant K_{d} of less than 40 μM as measured by surface plasmon resonance.
32. The recombinant polypeptide according to any of the preceding items, wherein said polypeptide is capable of binding to ILT2 or ILT4 with an affinity constant K_{d} of less than 20 μM as measured by surface plasmon resonance.
33. The recombinant polypeptide according to any of the preceding items, wherein said polypeptide is capable of binding to ILT2 or ILT4 with an affinity constant K_{d} of less than 10 μM as measured by surface plasmon resonance.
34. The recombinant polypeptide according to any of the preceding items, wherein said polypeptide is a dimer or multimer.
35. The recombinant polypeptide according to any of the preceding items, wherein said peptide antigen sequence according to i) is 7 to 11 amino acids in length, preferably 8-10 amino acids in length.
36. The recombinant polypeptide according to any of the preceding items, wherein the polypeptide comprises all of the components i) to vii) but preferably not components viii) to ix).
37. The recombinant polypeptide according to any of items 22 to 35, wherein the polypeptide comprises all of the components i) to ix).
38. The recombinant polypeptide according to any of the preceding items, further comprising an N-terminal secretion signal peptide sequence.
39. A pharmaceutical composition according to any of items 1 to 21, or a recombinant polypeptide according to any of items 22 to 38, for use in medicine.
40. A pharmaceutical composition according to any of items 1 to 21, or a recombinant polypeptide according to any of items 22 to 38, for use in a method for peptide antigen-specific immunomodulation in a subject, said immunomodulation being specific to the peptide antigen that is comprised by the pharmaceutical composition or recombinant polypeptide.
41. The pharmaceutical composition or recombinant polypeptide according to item 40 for the use according to item 40, wherein the method for immunomodulation is for inducing immunological tolerance towards the peptide antigen that is comprised by the pharmaceutical composition or recombinant polypeptide.
42. The pharmaceutical composition or recombinant polypeptide according to any of items 40-41 for the use according to any of items 40-41, wherein the method for immunomodulation is a method for the suppression of an immune autoimmune disease, for the suppression of an allergy, for the suppression of an immune reaction towards a biotherapeutical drug, for the suppression of an immune reaction towards an embryonic antigen, or for the suppression of an immune reaction towards transplanted cells, tissues or organs.
43. The pharmaceutical composition or recombinant polypeptide according to item 42 for the use according item 42, wherein the method for immunomodulation is a method for induction of immune tolerance and wherein the autoimmune disease affects multiple organs, hormone producing organs, nerves, joints, the skin, the gastrointestinal system, the eyes, blood components or blood vessels.
44. The pharmaceutical composition or recombinant polypeptide according to item 41 for the use according to item 41, wherein the method is a method for suppression of an immune response in Crohn's disease, ulcerative colitis, systemic lupus erythematosus (SLE), multiple sclerosis, rheumatoid arthritis, psoriasis, scleroderma, neuromyelitis optica or type 1 diabetes.
45. A nucleic acid encoding the polypeptide according to any one of items 22-38 or the polypeptide or MHC class Ib molecule as defined in any of items 1-21.
46. The nucleic acid according to item 45, wherein the nucleic acid is a vector.
47. A pharmaceutical composition comprising the nucleic acid according to items 45 or 46.
48. A recombinant host cell comprising a nucleic acid molecule or a vector according to item 45 or 46.
49. A method for producing a polypeptide according to any one of items 22-38, comprising culturing a recombinant host cell of item 48 under conditions allowing expression of the nucleic acid molecule, and recovering the polypeptide produced.
50. A combination of
   a1) an antigenic protein or peptide antigen, or a nucleic acid encoding said antigenic protein or peptide antigen, or an attenuated organism containing said antigenic protein or peptide antigen or a2) a cell presenting said peptide antigen according to a1);
   and
   b) an agent capable of blocking the binding between an MHC class Ib molecule and its receptor;
for use in a method of inducing in a human subject an immune response against said antigenic protein or peptide antigen.
51. The combination for use according to item 50, wherein the agent is capable of binding to said human MHC class Ib molecule and/or its receptors.
52. The combination for use according to any of the preceding items, wherein the agent is capable of binding to HLA-G.
53. The combination for use according to items 50-52, wherein the agent is an antibody, preferably a monoclonal antibody, which is capable of binding to HLA-G.
54. The combination for use according to any of the preceding items, wherein the agent is capable of binding to ILT2 or ILT4.
55. The combination for use according to any of the preceding items, wherein the agent is an antibody, preferably a monoclonal antibody, which is capable of binding to ILT2 or ILT4.
56. The combination for use according to any of the preceding items, wherein the agent comprises an Fc domain of an antibody or a fragment thereof.
57. The combination for use according to any of the preceding items, wherein the agent comprises an [alpha]3 domain of an MHC class Ib molecule.
58. The combination for use according to any of the preceding items, wherein the agent comprises one or more extracellular domains of ILT2 or ILT4 receptors, preferably at least the two N-terminal extracellular domains of ILT2 or ILT4 receptors, and wherein the agent comprises more preferably a soluble ILT2 or ILT4 receptor.
59. The combination for use according to any of the preceding items, wherein the agent is to be administered simultaneously with, before, or after administration of said antigenic protein or peptide antigen or said nucleic acid encoding said antigenic protein or peptide antigen or said attenuated organism containing said antigenic protein or peptide antigen.
60. The combination for use according to any of the preceding items, wherein the combination is a combination of a) an antigenic protein or peptide antigen; and b) an agent capable of blocking the binding between said MHC class Ib molecule and its receptor.
61. The combination for use according to any of items 50-59, wherein the combination is a combination of a) a nucleic acid encoding an antigenic protein or peptide antigen; and b) an agent capable of blocking the binding between said MHC class Ib molecule and its receptor.
62. The combination for use according to any of items 50-59, wherein the combination is a combination of a) an attenuated organism containing an antigenic protein or peptide antigen; and b) an agent capable of blocking the binding between said MHC class Ib molecule and its receptor.
63. The combination for use according to item 62, wherein the attenuated organism containing said antigenic protein or peptide antigen is an attenuated virus.
64. The combination for use according to any of items 50-62, wherein the antigenic protein or peptide antigen according to a) is a tumor antigen or an antigen that is at least 77% identical to the tumor antigen and is capable of inducing cross-protection against said antigen.
65. The combination for use according to any of the preceding items, wherein the method is a method for T cell based immunotherapy.
66. The combination for use according to any of items 50-63 and 65, wherein the antigenic protein or peptide antigen is detectable in pathogenic microorganisms or viruses.
67. The combination for use according to any of the preceding items, wherein the method is a method for the treatment or prevention of an infectious or malignant disease.
68. The combination for use according to item 67, wherein the disease is a cancer and wherein the peptide antigen is a tumor antigen.
69. The combination for use according to item 68, wherein the cancer is selected from the group consisting of melanoma, renal carcinoma, ovarian carcinoma, colorectal cancer, breast cancer, gastric cancer, pancreatic ductal adenocarcinoma, prostate cancer, B and T cell lymphoma and lung cancer.
70. The combination for use according to any of the preceding items, wherein the combination is present in one pharmaceutical composition.
71. The combination for use according to any of the preceding items, wherein said immune response against said antigenic protein or peptide antigen is specific to said antigenic protein or peptide antigen.
72. An agent capable of blocking the binding between an MHC class Ib molecule and its receptor as defined in any one of items 50 to 62, for use in a method for the treatment of a cancer in a human subject, said method including a therapy resulting in a release of cancer antigens from cells of said cancer.
73. The agent for use according to item 72, wherein said therapy resulting in a release of cancer antigens is chemotherapy or radiotherapy.
74. The pharmaceutical composition or recombinant polypeptide according to item 41 for the use according to item 41, wherein the method for inducing immunological tolerance towards the peptide antigen further comprises a peptide drug treatment, and wherein the peptide antigen is 1) identical to the peptide drug or is 2) a fragment of said peptide drug or is 3) a derivative of said fragment of said peptide drug that is capable of inducing immunological tolerance against said peptide drug.
75. The pharmaceutical composition or recombinant polypeptide according to item 41 for the use according to item 41, wherein the method for inducing immunological tolerance towards the peptide antigen further comprises a protein drug treatment, and wherein the peptide antigen is 1) a fragment of said protein drug or is 2) a derivative of said fragment of said protein drug that is capable of inducing immunological tolerance against said protein drug.
76. The pharmaceutical composition or recombinant polypeptide according to item 74 for the use according to item 74, wherein said peptide drug is to be administered in form of the peptide drug itself.
77. The pharmaceutical composition or recombinant polypeptide according to item 75 for the use according to item 75, wherein said protein drug is to be administered in form of the protein drug itself.
78. The pharmaceutical composition or recombinant polypeptide according to item 74 for the use according to item 74, wherein said peptide drug is to be administered by means of gene therapy, said gene therapy being a gene therapy with a gene encoding said peptide drug.
79. The pharmaceutical composition or recombinant polypeptide according to item 75 for the use according to item 75, wherein said protein drug is to be administered by means of gene therapy, said gene therapy being a gene therapy with a gene encoding said protein drug.

The invention may be used in any mammalian subject, preferably in human subjects.

Preferably, indications in which the above-mentioned combinations of immune-stimulatory T cell-directed treatments with blocking agents directed against MHC class Ib or ILT2/4 shall be used include viral infections and tumors in which elevated levels of HLA-G or other MHC class Ib molecules are detectable by methods such as polymerase chain reaction, ELISA, Western Blotting, immunofluorescence, immunohistochemistry and others (as described by Paul et al., Hum Immunol. 2000 November; 61(11):1177-95) in tumor effusions, blood samples, biopsies or other means on malignant cells or on non-malignant cells. As HLA-G is not expressed in many tissues but very potent even at low amounts, expression of a detectable level in an otherwise HLA-G deficient tissue or a 50% increase above the physiological level in a tissue which shows basal HLA-G expression is considered as a preferred elevated level in accordance with the invention.

### BRIEF DESCRIPTION OF THE DRAWINGS

FIG. 1: Cells expressing MHC Ib molecules loaded with defined peptides selectively eliminate CTLs specific for the presented peptide

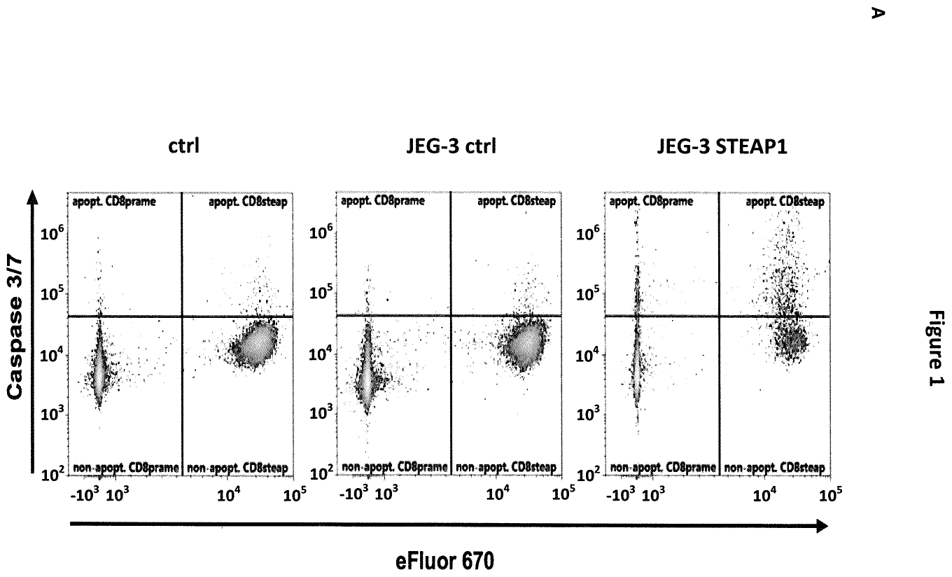

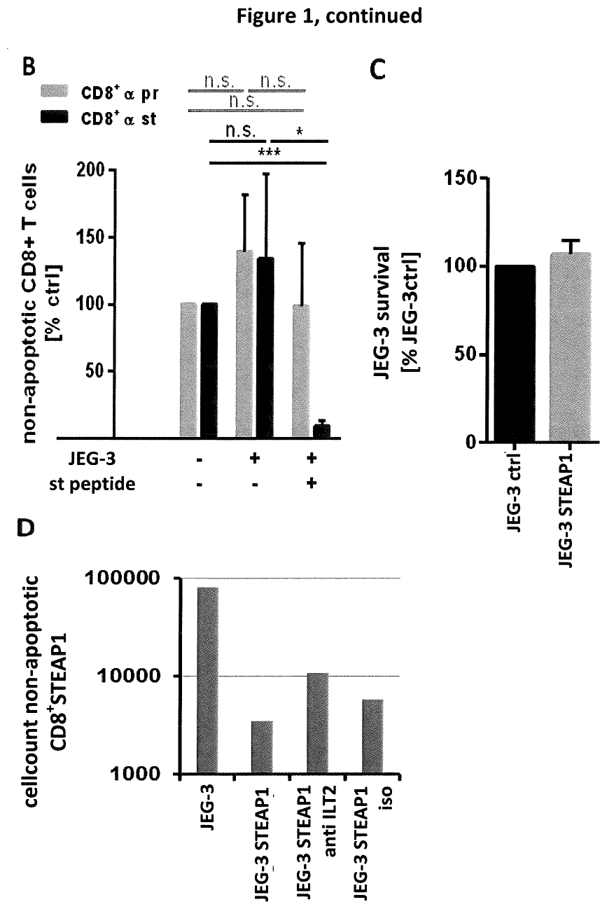

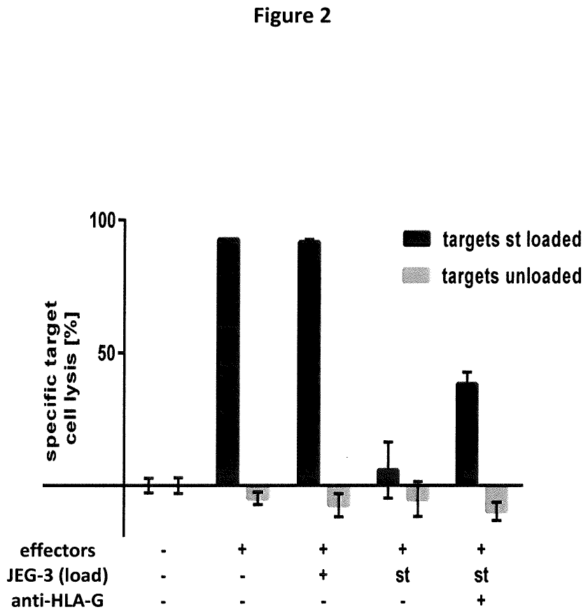

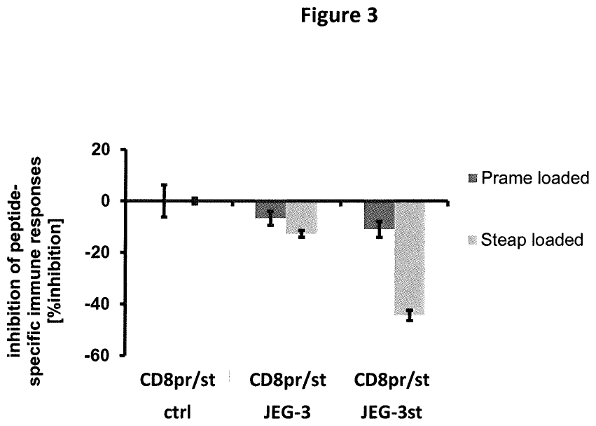

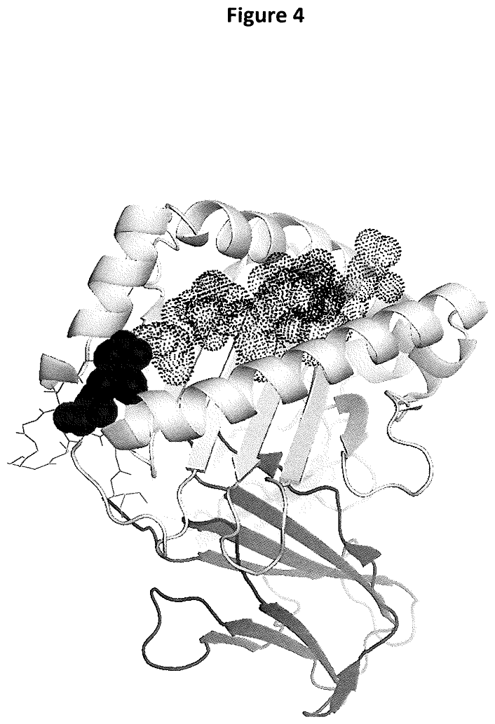

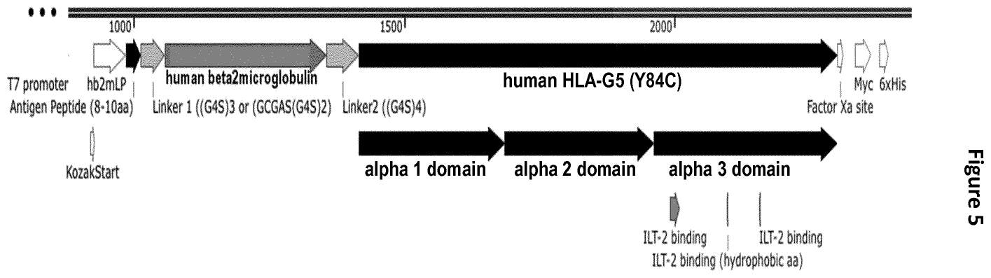

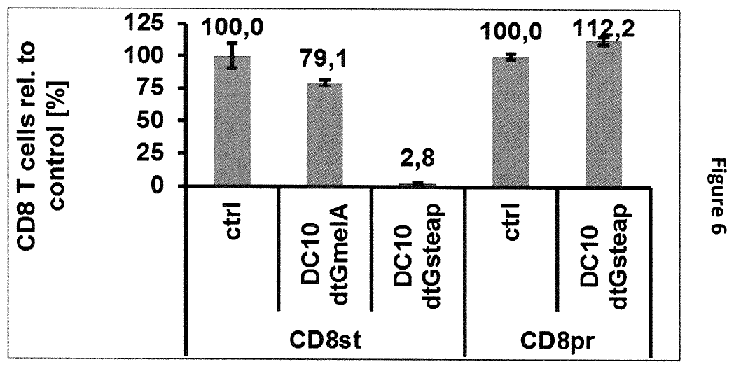

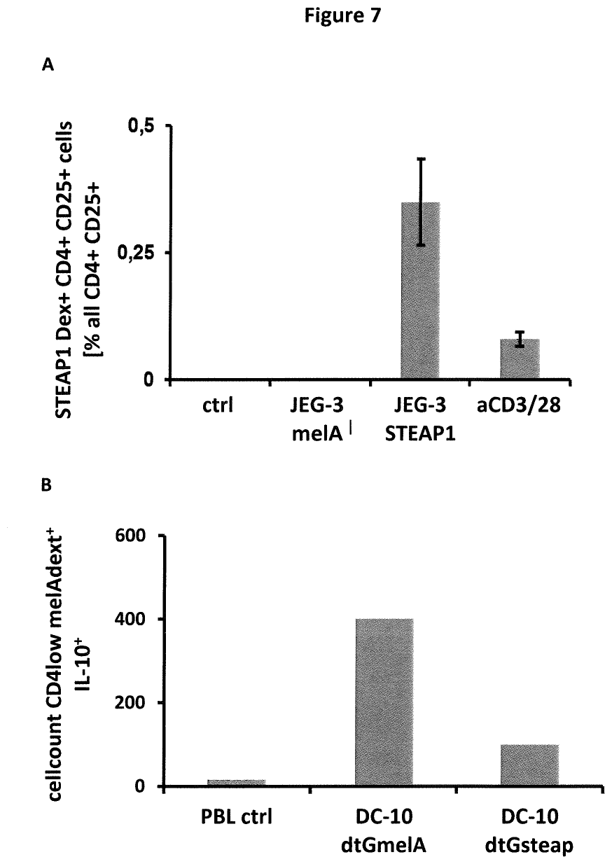

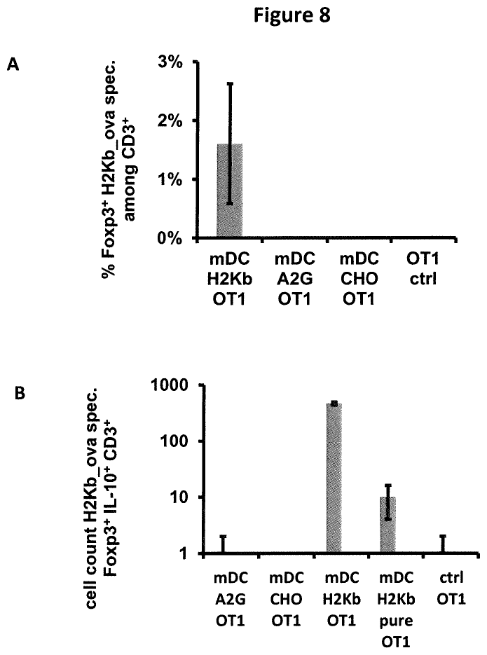

A, B) HLA-A2 restricted CD8⁺ effector T cells recognizing the model antigen STEAP1 (CD8st) can be selectively eliminated when their cognate peptide is presented on the tumor cell line JEG-3 which shows high expression of the non-classical MHC class Ib molecule HLA-G whereas classical MHC class Ia molecules are hardly detectable. Note that STEAP1 is generally also referred to herein as “STEAP”, “steap”, or abbreviated as “st”. These terms are used synonymously and are interchangeable. Co-cultured CD8⁺ effector T cells specific for the antigen PRAME (CD8pr) are not affected. (C) Despite loading with the cognate peptide for STEAP1-specific T cells, HLA-G expressing JEG-3 tumor cells are not eliminated during this process. This shows that MHC Ib molecules expressed by tumors or infected cells may render these cells resistant to targeting by antigen-specific T cells which normally are the main effectors of peptide-based vaccination strategies. (D) Induction of apoptosis in STEAP1-specific T cells by STEAP1 peptide presented on HLA-G can be strongly attenuated by a neutralizing antibody against the HLA-G interaction partner ILT-2 which is expressed on T cells.

FIG. 2: MHC Ib molecules loaded with defined peptides impair the cytotoxic potential of cognate CTLs in an antigen and HLA-G dependent manner.

HLA-A2-restricted T cell clones specific for STEAP1 or, respectively, PRAME were mixed and pretreated with control (+) or STEAP1-peptide loaded (st) JEG-3 cells. The neutralizing anti-human HLA-G antibody (clone 87G) was added at 10 μg/ml where indicated. After 16 h, the cytotoxic potential of the STEAP1 specific T cells towards luciferase-expressing naive (grey bars) or STEAP1-peptide loaded (black bars) HLA-A2⁺ UACC-257 melanoma cells was tested in a 2:1 ratio. After 8 h, D-luciferin was added and survival of target cells was determined in a luminometer using a biophotonic viability assay (Brown et al., J Immunol Methods. 2005 February; 297(1-2): 39-52.). HLA-G expressing JEG-3 cells reduced the lytic potential of STEAP1-specific CTLs by over 90% when loaded with STEAP1 peptide whereas naïve JEG-3 cells caused no significant inhibition. As this effect could be significantly attenuated by the presence of a partly neutralizing HLA-G antibody it can be concluded that peptide-loaded HLA-G can be used to inhibit T cell mediated immune reactions against selected antigens. According to the invention, this effect can be extended to further MHC class Ib molecules. Conversely, the induction of antigen-specific T cells mediated immune responses according to the invention can be achieved by agents that block MHC Ib.

FIG. 3: MHC Ib molecules combined with defined peptides inhibit cognate CTLs while immune responses towards other antigens remain largely unaffected

HLA-A2-restricted T cell clones specific for STEAP1 or, respectively, PRAME T cell clones specific for HLA-A2-STEAP1 and HLA-A2-PRAME were mixed and either left untreated (ctrl) or pretreated with control (JEG-3) or STEAP1-peptide loaded (JEG-3st) JEG-3 cells. After 8 h, the peptide-specific cytotoxic potential of both T cell clones towards luciferase-expressing PRAME-peptide (dark grey bars) or STEAP1-peptide loaded (light grey bars) luciferase expressing HLA-A2⁺ UACC-257 melanoma cells was tested in a 1:1 ratio. Pretreatment with STEAP1-peptide loaded JEG-3 cells inhibited the STEAP1 peptide specific T cell mediated immune response by about 50%, while the PRAME specific immune reaction remained largely unaltered by naïve or STEAP1-peptide loaded JEG-3 cells.

FIG. 4: Depiction of a peptide-loaded soluble MHC Ib molecule suitable to achieve therapeutic antigen-specific immunomodulation.

The presented peptide antigenis depicted in dotted spheres, the HLA-G alpha1-3 domains are sketched in light-grey, and the beta2microglobulin domain is shown in dark grey. An optional linker connecting the antigenic peptide with the beta2microglobulin molecule is displayed in grey stick style, and an optional disulfide trap is depicted in black spheres. This figure was generated using Pymol and is adapted from structures published in Clements et al., Proc Natl Acad Sci USA. 2005 Mar. 1; 102(9):3360-5 and Hansen et al., Trends Immunol. 2010 October; 31(10):363-9.

FIG. 5: Example for a vector-based construct encoding a single chain MHC Ib molecule suitable for therapeutic peptide-specific immunomodulation.

HLA-G1 and HLA-G5 each consist of 3 [alpha] domains (here in black), a non-covalently associated beta 2-microglobulin subunit (here in dark grey) and the antigenic peptide presented on HLA-G (short black arrow). HLA-G1 further contains a transmembrane domain and a short intracellular chain (not shown here). As shown here, the [alpha]-3 domain is capable of binding to the receptors ILT2 (see Shiroishi et al., Proc Natl Acad Sci USA. 2003 Jul. 22; 100(15):8856-8861) and ILT4 (see Shiroishi et al., Proc Natl Acad Sci USA. 2006 Oct. 31; 103(44):16412-7) on immune cells. Physiologically, these sequences form a non-covalently linked MHC class 1 complex. To simplify purification of the complex MHC Ib molecule, two protein tags (myc and His(6×)) were introduced in such a way as to enable their later removal via Factor Xa cleavage. Furthermore, the antigenic peptide, beta 2-microglobulin and MHC Ib [alpha]chain can be linked in order to increase the stability. The vector map was generated using Snapgene Viewer Software.

FIG. 6: Soluble peptide HLA-G/peptide-MHC Ib complexes combined with dendritic cells (DC-10) may selectively eliminate CD8⁺ effector T cells recognizing the presented target antigen.

Dendritic cells were generated from monocytes in the presence of GM-CSF, IL4 and IL10 (DC-10) before cell culture supernatants containing soluble peptide MHC Ib constructs were added for four hours. Disulfide-trap stabilized single chain HLA-G5 constructs encompassing presented Melan-A/MART1 (dtGmelA) or STEAP1 (dtGsteap) peptides were used. Binding of these constructs to DC-10 cells had been confirmed previously. Loaded DC-10 cells were then washed and cocultured for 48 h in a 1:1 ratio with control CTLs (PRAME specific, CD8pr) or target CTLs (STEAP1 specific, CD8st). These data suggest that dendritic cells loaded with soluble MHC Ib-peptide constructs can almost completely deplete cognate T cell clones whereas non-cognate CTLs are not affected.

FIG. 7: Peptide-loaded MHC Ib complexes induce human antigen-specific regulatory T cells recognizing the presented peptide

A) Peripheral blood mononuclear cells (PBMC) were taken from various healthy donors and co-cultured for 14 days in RPMI1640 medium with 5% hAB serum, 5 ng/ml TGFβ1, 20 ng/ml IL2 (Treg medium) with irradiated JEG-3 cells that had been loaded with Melan-A/MART1 (MART1)or STEAP1 (STEAP) peptides. At day 7, PBMCs were transferred to fresh medium and irradiated and peptide-loaded JEG-3 cells were added again. Treg expansion beads from Miltenyi Biotec (antiCD3, antiCD28 and antiCD2) were used as positive control. The obtained cells were stained with antibodies against human CD4 and CD25, with an HLA-A2 STEAP1 peptide dextramer (STEAP1 dex) and analyzed by flow cytometry. A significant enrichment of STEAP1 specific cells within the CD4⁺CD25^{high} Treg population was observed when STEAP1-loaded JEG-3 cells had been present.

B) 4×10⁵ DC-10 cells per well were loaded with disulfide trap single chain HLA-G constructs presenting a Melan-A (dtGmelA) or a STEAP1 (dtGsteap) peptide as described in FIG. 6. Then, 4×10⁶ PBL from the same donor were added, and cells were cultured for 7 days in Treg medium. Then, 4×10⁵ identically loaded DC-10 were added to each well. After a total of 14 days, Melan A specific IL-10 producing Treg cells were quantified by flow cytometry. The number of Melan A specific Treg was strongly increased in conditions in which PBL were cocultured with DC-10 loaded with single chain Melan A HLA-G molecules as compared to control molecules (STEAP1) or untreated PBL.

FIG. 8: Single chain peptide MHC constructs containing a human MHC Ib alpha3 domain in combination with DCs induce murine Treg cells specific for the presented peptide.

Murine DCs (mDCs) were generated by culturing bone marrow derived cells for 7 days in RPMI-1640 complete supplemented with 10% GM-CSF containing supernatant from Ag8653 myeloma cells transfected with the murine GM-CSF gene. mDCs were loaded with control CHO supernatants (CHO/ctrl) or supernatants from CHO cells transfected with plasmids coding for single chain peptide MHC molecules containing the human HLA-G alpha3 domain plus an ovalbumin peptide presented by murine H-2Kb alpha 1 and 2 domains (H2Kb). A similar construct containing the human HLA-A2 alpha 1 and 2 domains (A2G) instead of the murine H-2Kb alpha 1 and 2 domains was included as control. Expression of the constructs was confirmed by Western Blotting from the supernatants. A & B: Splenocytes from OT1 mice that express only a transgenic T cell receptor recognizing the H-2Kb presented OVA peptide (SIINFEKL) were cultured in Treg-permissive medium (RPMI complete, 5 ng/ml IL-2, 5 ng/ml TGF-β1) in a 2.5:1 ratio with loaded mDCs for 14 days.

### DETAILED DESCRIPTION OF THE INVENTION

### Definitions and General Techniques

Unless otherwise defined below, the terms used in the present invention shall be understood in accordance with their common meaning known to the person skilled in the art. All publications, patents and patent applications cited herein are hereby incorporated by reference in their entirety for all purposes.

All proteins in accordance with the invention, including the polypeptides and MHC molecules according to the invention, can be produced by methods known in the art. Such methods include methods for the production of recombinant proteins. It will be understood that the proteins in accordance with the invention, including the polypeptides and MHC molecules according to the invention, are meant to optionally include a secretion signal peptide sequence. Similarly, the proteins in accordance with the invention are meant to also optionally include affinity tags, e.g. in order to facilitate purification, and optional protease cleavage sites between the tag and the protein, e.g. in order to facilitate removal of the tags by protease cleavage.

Likewise, it will be understood that the proteins in accordance with the invention, including the polypeptides and MHC molecules according to the invention, are meant to include the respective pro-peptides.

It will also be understood that the polypeptides and MHC molecules according to the invention can be in form of their soluble or their membrane-bound form.

According to the invention, MHC molecules are preferably human MHC molecules.

The proteins and polypeptides of the invention, including the MHC molecules used according to the invention, the polypeptides of the invention and the antibodies in accordance with the invention, are preferably isolated.

The proteins and polypeptides of the invention, including the MHC molecules used according to the invention, the polypeptides of the invention and the antibodies in accordance with the invention, are preferably recombinant.

It will be understood how a polypeptide capable of binding and presenting an peptide antigen according to the invention can be prepared. For example, peptide antigen-binding domains such as [alpha]1 and [alpha]2 domains are well-known, and modifications of these domains can be made. The capability of a peptide antigen to bind to the polypeptides and MHC molecules according to the invention can be determined by techniques known in the art, including but not limited to explorative methods such as MHC peptide elution followed by Mass spectrometry and bio-informatic prediction in silico, and confirmative methods such as MHC peptide multimere binding methods and stimulation assays.

It will be understood that in connection with the peptide antigens used in accordance with the invention, any lengths of these peptide antigens referred to herein (e.g. “7 to 11 amino acids in length”) are meant to refer to the length of the peptide antigens themselves. Thus, the lengths of peptide antigens referred to herein do not include the length conferred by additional amino acids which are not part of the peptide antigens such as additional amino acids from possible linker sequences etc.

The term “autoimmune disease” is used herein in accordance with its common meaning known to the person skilled in the art and is not limited to particular autoimmune diseases. In accordance with all embodiments of the invention, autoimmune diseases are preferably autoimmune diseases which involve an autoimmune reaction to peptide autoantigens.

In accordance with the present invention, each occurrence of the term “comprising” may optionally be substituted with the term “consisting of”.

### Methods and Techniques

Generally, unless otherwise defined herein, the methods used in the present invention (e.g. cloning methods or methods relating to antibodies) are performed in accordance with procedures known in the art, e.g. the procedures described in Sambrook et al. (“Molecular Cloning: A Laboratory Manual.”, 2^{nd} Ed., Cold Spring Harbor Laboratory Press, Cold Spring Harbor, N.Y. 1989), Ausubel et al. (“Current Protocols in Molecular Biology.” Greene Publishing Associates and Wiley Interscience; New York 1992), and Harlow and Lane (“Antibodies: A Laboratory Manual” Cold Spring Harbor Laboratory Press, Cold Spring Harbor, N.Y. 1988), all of which are incorporated herein by reference.

Protein-protein binding, such as binding of antibodies to their respective target proteins, can be assessed by methods known in the art. Protein-protein binding, such as binding of antibodies to their respective target proteins, is preferably assessed by surface plasmon resonance spectroscopy measurements.

For instance, binding of MHC class Ib molecules or polypeptides according to the invention to their receptors, including ILT2 and ILT4, is preferably assessed by surface plasmon resonance spectroscopy measurements. More preferably, binding of MHC class Ib molecules or polypeptides according to the invention to their receptors is assessed by surface plasmon resonance measurements at 25° C. Appropriate conditions for such surface plasmon resonance measurements have been described by Shiroishi et al., Proc Natl Acad Sci USA. 2003 Jul. 22; 100(15):8856-8861.

Sequence Alignments of sequences according to the invention are performed by using the BLAST algorithm (see Altschul et al. (1990) “Basic local alignment search tool.” Journal of Molecular Biology 215. p. 403-410.; Altschul et al.: (1997) Gapped BLAST and PSI-BLAST: a new generation of protein database search programs. Nucleic Acids Res. 25:3389-3402.). Appropriate parameters for sequence alignments of short peptides by the BLAST algorithm, which are suitable for peptide antigens in accordance with the invention, are known in the art. Most software tools using the BLAST algorithm automatically adjust the parameters for sequence alignments for a short input sequence. In one embodiment, the following parameters are used: Max target sequences 10; Word size 3; BLOSUM 62 matrix; gap costs: existence 11, extension 1; conditional compositional score matrix adjustment. Thus, when used in connection with sequences, terms such as “identity” or “identical” preferably refer to the identity value obtained by using the BLAST algorithm.

### Preparation of Compositions of the Invention

Compositions in accordance with the present invention are prepared in accordance with known standards for the preparation of pharmaceutical compositions.

For instance, the compositions are prepared in a way that they can be stored and administered appropriately, e.g. by using pharmaceutically acceptable components such as carriers, excipients and/or stabilizers.

Such pharmaceutically acceptable components are not toxic in the amounts used when administering the pharmaceutical composition to a patient. The pharmaceutical acceptable components added to the pharmaceutical compositions may depend on the chemical nature of the active ingredients present in the composition, the particular intended use of the pharmaceutical compositions and the route of administration. In general, the pharmaceutically acceptable components used in connection with the present invention are used in accordance with knowledge available in the art, e.g. from Remington's Pharmaceutical Sciences, Ed. A R Gennaro, 20th edition, 2000, Williams & Wilkins, PA, USA.

Peptide Antigens in Accordance with the Invention

The peptide antigens which can be used in accordance with the invention, including the peptide antigens as defined above, are not particularly limited other than by their ability to be presented on MHC molecules.

Peptides which are able to be presented on MHC molecules can be generated as known in the art (see, for instance, Rammensee, Bachmann, Emmerich, Bachor, Stevanović. SYFPEITHI: database for MHC ligands and peptide motifs. Immunogenetics. 1999 November; 50(3-4): 213-9; Pearson et al. MHC class I-associated peptides derive from selective regions of the human genome. J Clin Invest. 2016 Dec. 1; 126(12):4690-4701; and Rock, Reits, Neefjes. Present Yourself! By MHC Class I and MHC Class II Molecules. Trends Immunol. 2016 November; 37(11):724-737).

Peptide antigens are generally known in the art. Generally, the peptide antigens in accordance with the invention are capable of binding to MHC class I proteins. It will be understood by a person skilled in the art that for each MHC class Ib molecule or polypeptide capable of presenting peptides in accordance with the invention, peptide antigens which are capable of binding to said MHC class Ib molecule or polypeptide will preferably be used. These peptide antigens can be selected based on methods known in the art.

Binding of peptide antigens to MHC class Ib molecules or to polypeptides capable of peptide antigen binding in accordance with the invention can be assessed by methods known in the art, e.g. the methods of:

Rammensee, Bachmann, Emmerich, Bachor, Stevanović. SYFPEITHI: database for MHC ligands and peptide motifs. Immunogenetics. 1999 November; 50(3-4): 213-9;

Pearson et al. MHC class I-associated peptides derive from selective regions of the human genome. J Clin Invest. 2016 Dec. 1; 126(12):4690-4701; and

Rock, Reits, Neefjes. Present Yourself! By MHC Class I and MHC Class II Molecules. Trends Immunol. 2016 November; 37(11):724-737.

Such methods include experimental methods and methods for the prediction of peptide antigen binding. Anchor residues which serve to anchor the peptide antigen on the MHC class I molecule and to ensure binding of the peptide antigen to the MHC class I molecule are known in the art.

In a preferred embodiment in accordance with all embodiments of the invention, the peptide antigen used in accordance with the invention contain any of the anchor or preferred amino acid residues in the positions as predicted for MHC class I molecules.

Such predictions can preferably be made in as described in any one of the following publications:

Rammensee et al, SYFPEITHI: database for MHC ligands and peptide motifs. Immunogenetics (1999) 50: 213-219

Nielsen et al, Protein Sci (2003) 12:1007-1017

Neefjes et al. Nat Rev Immunol. 2011 Nov. 11; 11(12):823-36

Diehl et al. Curr Biol. 1996 Mar. 1; 6(3):305-14,

Lee et al. Immunity. 1995 November; 3(5):591-600.

Desai & Kulkarni-Kale, T-cell epitope prediction methods: an overview. Methods Mol Biol. 2014; 1184:333-64.

In a preferred embodiment in accordance with all embodiments of the invention, the peptide antigen is from a human protein.

Alternatively, the non-anchor amino acid residues of the peptide antigen of the invention may be identical to the corresponding amino acid residues of a peptide antigen from a human protein, or they may have at least 50% sequence identity, preferably at least 60% sequence identity, more preferably at least 70% sequence identity, still more preferably at least 80% sequence identity, and still more preferably at least 90% sequence identity to the corresponding amino acid residues of a peptide antigen from a human protein. Alternatively, the non-anchor amino acid residues of the peptide antigen of the invention may contain conservative substitutions, preferably not more than two conservative substitutions, more preferably one conservative substitution with respect to the corresponding amino acid residues of a peptide antigen from a human protein. In a preferred embodiment, said human protein is a protein which expressed in tissues or cells that are affected by pathological immune reactions.

Peptide antigens in accordance with the invention can be naturally occurring peptides or non-naturally occurring peptides. Peptide antigens in accordance with the invention preferably consist of naturally occurring amino acids. However, non-naturally occurring amino acids such as modified amino acids can also be used. For instance, in one embodiment, the peptide antigens used in accordance with the invention can be peptidomimetics.

Methods for the synthesis of peptide antigens, including peptide antigens in accordance with the invention, are well known in the art.

### Sequences

Preferred amino acid sequences referred to in the present application can be independently selected from the following sequences. The sequences are represented in an N-terminal to C-terminal order; and they are represented in the one-letter amino acid code.

Leader Peptide: e.g. MSRSVALAVLALLSLSGLEA (SEQ ID No: 1)
Peptide antigen: any MHC class I peptide corresponding to MHC class I [alpha] 1&2 domains, e.g.
MLAVFLPIV (STEAP1) (SEQ ID No: 2) or SIINFEKL (Ova) (SEQ ID No: 3)
Linker1 (disulfide trap stabilized): For instance GGGGSGGGGSGGGGS (SEQ ID No: 4) or
GCGASGGGGSGGGGS (SEQ ID No: 5)
beta 2 Microglobulin derived from human or other-species, for instance:
IQRTPKIQVYSRHPAENGKSNFLNCYVSGFHPSDIEVDLLKNGERIEKVEHSDLSFSKDWSFYLLYYTEFTPTE
KDEYACRVNHVTLSQPKIVKWDRDM (SEQ ID No: 6, human beta 3 Microglobulin)
Linker2, for instance
GGGGSGGGGSGGGGSGGGGS (SEQ ID No: 7)
[Alpha] 1 & 2 domain derived either from human HLA-G or from any other MHC class I [alpha]1&2
domain suitable to present the selected antigenic peptide, Y84 may be C in DT variant
GSHSMRYFSAAVSRPGRGEPRFIAMGYVDDTQFVRFDSDSACPRMEPRAPWVEQEGPEYWEEETRNTKAH
AQTDRMNLQTLRGYYNQSEASSHTLQWMIGCDLGSDGRLLRGYEQYAYDGKDYLALNEDLRSWTAADTAA
QISKRKCEAANVAEQRRAYLEGTCVEWLHRYLENGKEMLQRA (SEQ ID No: 8)
e.g.: Murine H2Kb [alpha]1 & 2 domain (Y840)
GPHSLRYFVTAVSRPGLGEPRYMEVGYVDDTEFVRFDSDAENPRYEPRARWMEQEGPEYWERETQMKAKG
NEQSFRVDLRTLLGCYNQSKGGSHTIQVISGCEVGSDGRLLRGYQQYAYDGCDYIALNEDLKTWTAADMAAL
ITKHKWEQAGEAERLRAYLEGTCVEWLRRYLKNGNATLLRT (SEQ ID No: 9)
Or: Human HLA-A2 [alpha]1 & 2 domain
GSHSMRYFFTSVSRPGRGEPRFIAVGYVDDTQFVRFDSDAASQRMEPRAPWIEQEGPEYWDGETRKVKAH
SQTHRVDLGTLRGYYNQSEAGSHTVQRMYGCDVGSDWRFLRGYHQYAYDGKDYIALKEDLRSWTAADMAA
QTTKHKWEAAHVAEQLRAYLEGTCVEWLRRYLENGKETLQRT (SEQ ID No: 10)
Human HLA-G [alpha]3 domain (or any MHC lb [alpha]3 domain, such as HLA-F, which also interacts
with ILT2 and ILT4 receptors), for instance:
DPPKTHVTHHPVFDYEATLRCWALGFYPAEIILTWQRDGEDQTQDVELVETRPAGDGTFQKWAAVVVPSGE
EQRYTCHVQHEGLPEPLMLRWSKEGDGGIMSVRESRSLSEDL (SEQ ID No: 11; sequence of HLA-G
[alpha]3). Note that the following underlined amino acids of this sequence are relevant for ILT2
or IL14 receptor interaction:
DPPKTHVTHHPVFDYEATLRCWALGFYPAEIILTWQRDGEDQTQDVELVETRPAGDGTFQKWAAVVVPSGE
EQRYTCHVQHEGLPEPLMLRWSKEGDGGIMSVRESRSLSEDL
Factor Xa restriction site: IEGRTGTKLGP (SEQ ID No: 12)
Myc tag: EQKLISEEDL (SEQ ID No: 13)
Additional sequence: NSAVD
His tag: HHHHHH* (SEQ ID No: 14)
Examples for mature full length proteins of the invention:
disulfide trap_Ova_Linker1_humanbeta2microglobulin_Linker2_H2Kbalpha1&2_HLA-
Galpha3_XaSite_myc&hisTAG
(dtH2Gova)
SIINFEKLGCGASGGGGSGGGGSIQRTPKIQVYSRHPAENGKSNFLNCYVSGFHPSDIEVDLLKNGERIEKVE
HSDLSFSKDWSFYLLYYTEFTPTEKDEYACRVNHVTLSQPKIVKWDRDMGGGGSGGGGSGGGGSGGGGS
GPHSLRYFVTAVSRPGLGEPRYMEVGYVDDTEFVRFDSDAENPRYEPRARWMEQEGPEYWERETQKAKG
NEQSFRVDLRTLLGCYNQSKGGSHTIQVISGCEVGSDGRLLRGYQQYAYDGCDYIALNEDLKTWTAADMAAL
ITKHKWEQAGEAERLRAYLEGTCVEWLRRYLKNGNATLLRTDPPKTHVTHHPVFDYEATLRCWALGFYPAEII
LTWQRDGEDQTQDVELVETRPAGDGTFQKWAAVVVPSGEEQRYTCHVQHEGLPEPLMLRWSKEGDGGIM
SVRESRSLSEDLIEGRTGTKLGPEQKLISEEDLNSAVDHHHHHH* (SEQ ID No: 15)
Note that the sequence of the peptide antigen (here: SIINFEKL) of the above full length protein
can be substituted by any peptide antigen sequence in accordance with the invention.
disulfidetrap_STEAP1_Linker1_humanbeta2microglobulin_Linker2_HLA-A2alpha1&2_HLA-
Galpha3_XaSite_myc&hisTAG
(dtGsteap)
MLAVFLPIVGCGASGGGGSGGGGSIQRTPKIQVYSRHPAENGKSNFLNCYVSGFHPSDIEVDLLKNGERIEKV
EHSDLSFSKDWSFYLLYYTEFTPTEKDEYACRVNHVTLSQPKIVKWDRDMGGGGSGGGGSGGGGSGGGGS
GSHSMRYFSAAVSRPGRGEPRFIAMGYVDDTQFVRFDSDSACPRMEPRAPWVEQEGPEYWEEETRNTKAH
AQTDRMNLQTLRGCYNQSEASSHTLQWMIGCDLGSDGRLLRGYEQYAYDGKDYLALNEDLRSWTAADTAA
QISKRKCEAANVAEQRRAYLEGTCVEWLHRYLENGKEMLQRADPPKTHVTHHPVFDYEATLRCWALGFYPA
EIILTWQRDGEDQTQDVELVETRPAGDGTFQKWAAVVVPSGEEQRYTCHVQHEGLPEPLMLRWSKEGDGGI
MSVRESRSLSEDLIEGRTGTKLGPEQKLISEEDLNSAVDHHHHHH* (SEQ ID No: 16)
Note that the sequence of the peptide antigen (here: MLAVFLPIV) of the above full length protein
can be substituted by any peptide antigen sequence in accordance with the invention.
The receptors ILT2 (also known as LILRB1) and ILT4 (also known as LILRB2) are known in the art.
Preferred sequences of these receptors in accordance with the invention are as follows:
ILT2:
MTPILTVLICLGLSLGPRTHVQAGHLPKPTLWAEPGSVITQGSPVTLRCQGGQETQEYRL
YREKKTALWITRIPQELVKKGQFPIPSITWEHAGRYRCYYGSDTAGRSESSDPLELVVTG
AYIKPTLSAQPSPVVNSGGNVILQCDSQVAFDGFSLCKEGEDEHPQCLNSQPHARGSSRA
IFSVGPVSPSRRWWYRCYAYDSNSPYEWSLPSDLLELLVLGVSKKPSLSVQPGPIVAPEE
TLTLQCGSDAGYNRFVLYKDGERDFLQLAGAQPQAGLSQANFTLGPVSRSYGGQYRCYGA
HNLSSEWSAPSDPLDILIAGQFYDRVSLSVQPGPTVASGENVTLLCQSQGWMQTFLLTKE
GAADDPWRLRSTYQSQKYQAEFPMGPVTSAHAGTYRCYGSQSSKPYLLTHPSDPLELVVS
GPSGGPSSPTTGPTSTSGPEDQPLTPTGSDPQSGLGRHLGVVIGILVAVILLLLLLLLLF
LILRHRRQGKHWTSTQRKADFQHPAGAVGPEPTDRGLQWRSSPAADAQEENLYAAVKHTQ
PEDGVEMDTRSPHDEDPQAVTYAEVKHSRPRREMASPPSPLSGEFLDTKDRQAEEDRQMD
TEAAASEAPQDVTYAQLHSLTLRREATEPPPSQEGPSPAVPSIYATLAIH (SEQ ID No: 17)
ILT4:
MTPIVTVLICLGLSLGPRTHVQTGTIPKPTLWAEPDSVITQGSPVTLSCQGSLEAQEYRL
YREKKSASWITRIRPELVKNGQFHIPSITWEHTGRYGCQYYSRARWSELSDPLVLVMTGA
YPKPTLSAQPSPVVTSGGRVTLQCESQVAFGGFILCKEGEEEHPQCLNSQPHARGSSRAI
FSVGPVSPNRRWSHRCYGYDLNSPYVWSSPSDLLELLVPGVSKKPSLSVQPGPVVAPGES
LTLQCVSDVGYDRFVLYKEGERDLRQLPGRQPQAGLSQANFTLGPVSRSYGGQYRCYGAH
NLSSECSAPSDPLDILITGQIRGTPFISVQPGPTVASGENVTLLCQSWRQFHTFLLTKAG
AADAPLRLRSIHEYPKYQAEFPMSPVTSAHAGTYRCYGSLNSDPYLLSHPSEPLELVVSG
PSMGSSPPPTGPISTPAGPEDQPLTPTGSDPQSGLGRHLGVVIGILVAVVLLLLLLLLLF
LILRHRRQGKHWTSTQRKADFQHPAGAVGPEPTDRGLQWRSSPAADAQEENLYAAVKDTQ
PEDGVEMDTRAAASEAPQDVTYAQLHSLTLRRKATEPPPSQEREPPAEPSIYATLAIH (SEQ ID No: 18)

The present invention is further illustrated by the following non-limiting examples:

### EXAMPLES

### General Notes

All steps were carried out under sterile conditions; protective containers were only opened under laminar flow hoods. Cells were always centrifuged at 350×g for 5 minutes unless indicated otherwise. All viable cells were maintained in incubators at 37° C., 5% CO₂ and >95% humidity. A water bath set to 37° C. was used to prewarm media, PBS or other solutions added to the cells. Neubauer chambers were used for cell counting. Student's T-Test was used for statistical analysis, p values below 0.05 were considered significant.

### Example 1: Cells Expressing MHC Ib Molecules Loaded with Defined Peptides Selectively Eliminate CTLs Specific for the Presented Peptide

### Materials and Methods:

JEG-3 is a human choriocarcinoma cell line expressing high levels of HLA-G and hardly any classical MHC class I molecules (Rinke de Wit et. al., J Immunol. 1990 Feb. 1; 144(3):1080-7). JEG-3 cells were cultured in complete RPMI1640 medium with 10% fetal calf serum, 0.5% sodium pyruvate solution (100 mM) and 1% penicillin (10 kU/ml) and streptomycin (10 mg/ml) solution, (“RPMI complete”). 3×10⁵ JEG-3 cells were seeded in 1 ml RPMI1640 complete in 12 well-plates.

Where indicated, 1 μl of stock solution with STEAP1 (292.2L-9mer, MLAVFLPIV) or PRAME (435-9mer, NLTHVLYPV) peptides (5 μg/μl) was added. The next day, JEG-3 cells were washed three times with PBS before 300 μl supplemented CellGro DC medium (5% human serotype AB serum, 25-50 U/ml IL-2, 5 ng/ml IL-15) were added to each well.

Clonal HLA-A*02 (HLA-A2) restricted, STEAP1 (st) or PRAME (pr) peptide-specific CD8⁺ T cells (STEAP1-/PRAME “specific”) were generated according to Wölfl et al, Nat Protoc. 2014 April; 9(4):950-66. STEAP1-specific CD8+ T cells are stained with Cell Proliferation Dye eFluor® 670 according to the manufacturers instructions and resuspended in complete RPMI1640 medium which has been described above. 1.5×10⁵ cells in 300 μl of medium are added to each well with peptide-loaded JEG-3. In the same manner, unstained PRAME-specific CD8⁺ T cells were pelleted, resuspended and added to each well. In the experiment shown in D, anti-ILT-2 antibody (clone HP-F1) or isotype control antibody was added to a final concentration of 10 μg/ml.

After 16 hours, the cells were collected and stained with 5 μM CellEvent Caspase-3/7 Green (Life Technologies) according to the manufacturers instructions. Non-adherent cells were then collected and stained for 30 min on ice in 1:100 dilutions of anti-human CD8 (PE/Cy7, clone RPA-T8) and anti-human CD4 (PE/Dye647, clone MEM-241) antibodies and analyzed by flow cytometry. As CTLs are CD8⁺CD4⁻, CD4 staining enabled the exclusion of possible CD4⁺/CD8⁺ double-positive cells and autofluorescent cells. The total cell numbers were determined based on cell counts per μl. Survival of the adherent JEG-3 cells was quantified by crystal violet assay.

### Results:

A) In both control conditions without JEG-3 cells or with HLA-G⁺ DMSO treated control JEG-3 cells less than 5% apoptotic, Caspase 3/7^{+ eFluor}670⁻ PRAME-specific or eFluor670⁺ STEAP1-specific CD8⁺ T cells were detected. In contrast, after coculture with STEAP1-loaded JEG-3 cells, more than 90% of the STEAP1-specific CD8⁺ T cells are eliminated or apoptotic, while no significant effects on PRAME-specific T cells were observed. STEAP1-specific CD8⁺ T cells were easily distinguishable from PRAME-specific T cells due to the bright eFluor 670 staining. This dotplot is a representative result from one of three experiments. B) Statistical analysis of three independent experiments shows that these effects are highly significant, and that STEAP1-specific T cells can be selectively eliminated in coculture with HLA-G⁺ JEG-3 cells that are loaded with the cognate peptide. C) JEG-3 cell survival is not reduced due to loading with peptides recognized by the cocultured T cells. D) Under the same conditions, the addition of an antibody that blocks the HLA-G receptors ILT2 partially inhibited targeted elimination of STEAP1-specific T cells.

### Conclusion:

This experiment shows that peptide-specific CD8⁺ T cells can be selectively eliminated if they are in contact with human MHC Ib⁺ cells such as JEG-3 cells presenting their cognate antigen. This is surprising, as MHC Ia⁺ target cells that present cognate peptides to activated CD8⁺ T cells are usually eliminated while the T cells survive. In contrast to MHC Ia⁺ targets, peptide-loading of JEG-3 cells did not result in reduced survival, indicating that MHC Ib molecules may have opposing effects as compares to MHC Ia molecules. Furthermore, MHC Ib molecules and their receptor ILT2 cooperate to achieve this effect, as shown by the inhibition of this effect which was achieved by agents blocking their interaction, such as ILT2 blocking antibodies. Therefore, according to the invention, such blocking agents can be used to promote the induction of peptide-specific immune responses in the presence of MHC Ib molecules.

### Example 2: MHC Ib Molecules Loaded with Defined Peptides can be Used to Inhibit the Cytotoxic Potential of Cognate CTLs in an Antigen-Specific Manner

### Materials and Methods:

1×10⁶ JEG-3 cells were either left untreated or loaded with STEAP1 peptide (“st”, see example 1) in 1 ml RPMI1640 complete in 6 well-plates. 5×10⁵ STEAP1-specific CD8⁺ T cells were mixed with 5×10⁵ PRAME-specific CD8⁺ T cells (effectors) and left untreated or co-cultured with these JEG-3 for 16 h. 10 μg/ml of the neutralizing anti-human HLA-G antibody (clone 87G, BioLegend, Germany) was added where indicated. On the next day, firefly luciferase expressing HLA-A2⁺ UACC-257 melanoma cells (targets) were detached using accutase solution (PAA, Germany), washed and loaded with STEAP1 peptide (5 μg/ml, “st loaded”) or equivalent amounts of DMSO (“unloaded”) on a shaker at 37° C. for 4 h. 1×10⁴ UACC cells per well were then seeded in a white round bottom 96 well plate. The non-adherent mixed T cells were then collected, and an equivalent of 4×10⁴ initial T cells (2×10⁴ each) and firefly D-luciferin (PJK Germany, final concentration 140 μg/ml) were added. Target cell survival was determined in a luminometer after 8 h (method details Brown et al., J Immunol Methods. 2005 February; 297(1-2): 39-52.).

### Results:

Presentation of a peptide antigen on HLA-G+ JEG-3cells impaired the cytotoxic capacity of CD8⁺ T cell clones recognizing this specific peptide antigen in an MHC-lb dependent manner. In the described setting, STEAP1 specific control CTLs or CTLs pretreated with HLA-G+ JEG-3 cells lysed about 90% of all target cells loaded with the cognate peptide, while naive target cells were not eliminated. In contrast, pretreatment with JEG-3 cells and the cognate peptide almost completely protected the antigen-presenting target cells. An antibody which can partially block HLA-G dependent effects (87G) partially reverted this peptide-specific immunosuppressive effect. This implies that peptide-loaded MHC class Ib molecules could also suppress unwanted cytotoxic (auto)immune reactions against the presented antigen in a clinical setting.

Furthermore, MHC Ib positive tumour cells that are in contact with peptides (e.g. through radiation, chemotherapy or peptide-vaccination regimen) may specifically suppress CD8⁺ T cell-mediated anti-tumour immune responses. This effect, however, can be abrogated by agents that block the interaction between MHC Ib molecules and their receptors.

### Example 3: MHC Ib Molecules Combined with Defined Peptides Inhibit Cognate CTLs while Immune Responses Towards Other Antigens Remain Largely Unaffected

### Materials and Methods:

In the experiment shown in FIG. 3, HLA-A2 STEAP1-specific (CD8st) and PRAME-specific CD8⁺ T cells (CD8pr) were mixed and either left untreated or co-cultured with JEG-3 cells loaded or not with STEAP1 peptide for 8 h (methods see FIG. 2). The T cells in suspension were then collected and combined with luciferase-expressing PRAME-peptide (dark grey bars) or STEAP1-peptide loaded (light grey bars) HLA-A2³⁰ UACC-257 melanoma cells (T cells not counted after pretreatment, initial effector:target ratio 1:1).

### Results:

Pre-exposing the mixed CD8 T cell clones to one of the cognate peptides in context of an MHC Ib positive cell line reduced the cytotoxic potential of the cognate T cells to about 50%, while the cytotoxic activity of the other T cell clone remained at about 90%, which was comparable to the peptide-independent immunosuppressive the effect of HLA-G⁺ JEG-3 cells alone. Consequently, this approach shows that tolerance can be induced against a specific (auto)immune-relevant target antigen without simultaneously undermining desirable immune responses against different (e.g. viral) antigens. Based on the MHC pattern displayed by JEG-3 cells and on the previously shown experiments with neutralizing antibodies it will be understood that these peptide-specific effects are mediated via HLA-G. This experiment implies that presentation of an antigenic peptide on MHC Ib molecules can impair the cytolytic capacity of cognate CD8⁺ T cells.

### Example 4: Building Plan for Therapeutic Agent: Soluble Single Chain Construct Containing Antigenic Peptide, MHC Class 1-Based [Alpha]1 and [Alpha]2 Domain, HLA-G (or Other MHC Class Ib Molecule)-Derived [Alpha]3 Domain and [Beta]2-Microglobulin

### Design of MHC Ib Peptide Complexes

MHC class Ib molecules like HLA-G naturally consist of three polypeptide molecules in one complex. As shown in FIG. 4, these may be linked by linkers in order to improve the stability.

Alternatively, all components can be displayed in a linear manner, as shown in FIG. 5.

Sequences as used in specific embodiments are listed below.

Components of the Coding Sequence:

Leader Peptide: e.g. secretion inducing leader
peptides such as
(SEQ ID No: 1)
MSRSVALAVLALLSLSGLEA
Presented peptide antigen: any peptide of 8 to 12
amino acids possessing anchor residues that allow
for presentation by MHC class I [alpha] 1&2
domains, e.g.
(SEQ ID No: 2)
MLAVFLPIV (STEAP1)
or
(SEQ ID No: 3)
SIINFEKL (Ova)
Linker1 (disulfide trap stabilized):
(SEQ ID No: 4)
GGGGSGGGGSGGGGS
or
(SEQ ID No: 5)
GCGASGGGGSGGGGS
beta 2 Microglobulin derived from human or other-
species
(SEQ ID No: 6)
IQRTPKIQVYSRHPAENGKSNFLNCYVSGFHPSDIEVDLLKNGERIEKVEH
SDLSFSKDWSFYLLYYTEFTPTEKDEYACRVNHVTLSQPKIVKWDRDM
Linker2
(SEQ ID No: 7)
GGGGSGGGGSGGGGSGGGGS
[Alpha] 1 & 2 domain derived either from human
HLA-G or from any other MHC class I [alpha]1&2
domain suitable to present the selected
antigenic peptide, Y84 may be C in DT variant
(SEQ ID No: 8)
GSHSMRYFSAAVSRPGRGEPRFIAMGYVDDTQFVRFDSDSACPRMEPRAPW
VEQEGPEYVVEEETRNTKAHAQTDRMNLQTLRGYYNQSEASSHTLQWMIGC
DLGSDGRLLRGYEQYAYDGKDYLALNEDLRSWTAADTAAQISKRKCEAANV
AEQRRAYLEGTCVEWLHRYLENGKEMLQRA
e.g.: Murine H2Kb [alpha]& 2 domain (Y84C)
(SEQ ID No: 9)
GPHSLRYFVTAVSRPGLGEPRYMEVGYVDDTEFVRFDSDAENPRYEPRARW
MEQEGPEYWERETQKAKGNEQSFRVDLRTLLGCYNQSKGGSHTIQVISGCE
VGSDGRLLRGYQQYAYDGCDYIALNEDLKTWTAADMAALITKHKWEQAGEA
ERLRAYLEGTCVEWLRRYLKNGNATLLRT
Or:
Human HLA-A2 [alpha]1 & 2 domain
(SEQ ID No: 10)
GSHSMRYFFTSVSRPGRGEPRFIAVGYVDDTQFVRFDSDAASQRMEPRAPW
IEQEGPEYWDGETRKVKAHSQTHRVDLGTLRGYYNQSEAGSHTVQRMYGCD
VGSDWRFLRGYHQYAYDGKDYIALKEDLRSWTAADMAAQTTKHKWEAAHVA
EQLRAYLEGTCVEWLRRYLENGKETLQRT
Human HLA-G [alpha]3 domain (or any MHC lb [alpha]3
domain, such as HLA-F, which also interacts with
ILT2 and ILT4 receptors, underlined amino acids are
relevant for interaction with ILT-2 or ILT-4), for
example
(SEQ ID No: 11)
DPPKTHVTHHPVFDYEATLRCWALGFYPAEIILTWQRDGEDQTQDVELVET
RPAGDGTFQKWAAVVVPSGEEQRYTCHVQHEGLPEPLMLRWSKEGDGGIMS
VRESRSLSEDL
Factor Xa restriction site:
(SEQ ID No: 12)
IEGRTGTKLGP
Myc tag:
(SEQ ID No: 13)
EQKLISEEDL
Additional sequence:
NSAVD
His tag:
(SEQ ID No: 14)
HHHHHH*
Examples for mature full length proteins:
Disulfide trap_Ova_Linked_humanbeta2micro-
globulin_Linker2_H2Kbalpha1&2_HLA-
Galpha3_XaSite_myc&hisTAG
(dtH2KbGova)
(SEQ ID No: 15)
SIINFEKLGCGASGGGGSGGGGSIQRTPKIQVYSRHPAENGKSNFLNCYVS
GFHPSDIEVDLLKNGERIEKVEHSDLSFSKDWSFYLLYYTEFTPTEKDEYA
CRVNHVTLSQPKIVKWDRDMGGGGSGGGGSGGGGSGGGGSGPHSLRYFVTA
VSRPGLGEPRYMEVGYVDDTEFVRFDSDAENPRYEPRARWMEQEGPEYWER
ETQKAKGNEQSFRVDLRTLLGCYNQSKGGSHTIQVISGCEVGSDGRLLRGY
QQYAYDGCDYIALNEDLKTWTAADMAALITKHKWEQAGEAERLRAYLEGTC
VEWLRRYLKNGNATLLRTDPPKTHVTHHPVFDYEATLRCWALGFYPAEIIL
TWQRDGEDQTQDVELVETRPAGDGTFQKWAAVVVPSGEEQRYTCHVQHEGL
PEPLMLRWSKEGDGGIMSVRESRSLSEDLIEGRTGTKLGPEQKLISEEDLN
SAVDHHHHHH*
disulfidetrap_STEAP1_Linker1_humanbeta2micro-
globulin_Linker2_HLA-A2alpha1&2_HLA-
Galpha3_XaSite_myc&hisTAG
(dtGsteap)
(SEQ ID No: 16)
MLAVFLPIVGCGASGGGGSGGGGSIQRTPKIQVYSRHPAENGKSNFLNCYV
SGFHPSDIEVDLLKNGERIEKVEHSDLSFSKDWSFYLLYYTEFTPTEKDEY
ACRVNHVTLSQPKIVKWDRDMGGGGSGGGGSGGGGSGGGGSGSHSMRYFSA
AVSRPGRGEPRFIAMGYVDDTQFVRFDSDSACPRMEPRAPWVEQEGPEYWE
EETRNTKAHAQTDRMNLQTLRGCYNQSEASSHTLQWMIGCDLGSDGRLLRG
YEQYAYDGKDYLALNEDLRSWTAADTAAQISKRKCEAANVAEQRRAYLEGT
CVEWLHRYLENGKEMLQRADPPKTHVTHHPVFDYEATLRCWALGFYPAEII
LTWQRDGEDQTQDVELVETRPAGDGTFQKWAAVVVPSGEEQRYTCHVQHEG
LPEPLMLRWSKEGDGGIMSVRESRSLSEDLIEGRTGTKLGPEQKLISEEDL
NSAVDHHHHHH*

### Example 5: Soluble Peptide-MHC Ib Complexes Combined with Dendritic Cells (DC-10) May Selectively Eliminate CD8⁺ Effector T Cells Recognizing the Presented Target Antigen

### Materials and Methods:

In order to investigate whether soluble peptide MHC Ib constructs can eliminate effector T cells in an antigen dependent manner these constructs were loaded on dendritic cells expanded in the presence of IL-4, GM-CSF and IL-10 (DC-10). DC-10 were generated by culturing 5×10⁶ MACS purified (CD14 beads, Miltenyi, Germany) CD14⁺ cells from healthy donors per ml for 7 days in DC-10-Medium (complete RPMI1640 medium, 10 ng/ml IL-4, 10 ng/ml IL-10, 100 ng/ml GM-CSF). New medium was added on days 3 and 5. The obtained DC-10 cells did not adhere to the cell culture dish. 4×10⁵ DC-10 cells per ml were then combined with an equivalent amount of day 5 cell culture supernatants from CHO cells (1×10⁶/ml) transiently transfected by Lipofection with pCDNA3.1 expression vectors for single chain disulfide trapped peptide HLA-G constructs containing a STEAP1 peptide (dtGsteap, sequence see Example 4) or a Melan A/MART-1 peptide (ELAGIGILTV, dtGmelA) or control supernatant for 4 h. DC-10 were then washed with PBS 3 times and resuspended in 50 μl RPMI 1640 medium with 5 hAB serum+IL-2 (10⁶ DC-10/ml). 5×10⁴ peptide-MHC Ib loaded DC-10 cells were then combined with HLA-A2 restricted, antigen-specific CD8⁺ T cells recognizing either STEAP1 (CD8st) or PRAME (CD8pr) in a 1:1 ratio for 16 h. Cells were then stained with CellEvent Caspase-3/7 Green (5 μM, Life Technologies) according to the manufacturer's instructions and antibodies specific for human CD4 (clone EDU-2) and CD8 (clon RPA-T8) (see example 2). CD8⁺CD4⁻ caspase3/7⁻ cells were quantified by flow cytometry.

### Results:

As shown in FIG. 6, in two independent experiments, STEAP1 specific T cells combined with DC-10 cells loaded with single chain MHC Ib constructs presenting the cognate peptide were almost completely eliminated within 16 h. The same conditions did not negatively affect the survival of T cells specific for a control peptide (CD8pr). Contructs containing a control peptide (dtGmelA) only slightly reduced the survival of STEAP1 specific CD8⁺ T cells. This indicates that also soluble MHC Ib molecules combined with specific peptides can be used to selectively eliminate effector T cells specific for the presented peptide and thus to selectively modulate immune responses to defined antigens.

### Example 6: Peptide-Loaded MHC Ib Complexes Induce Human Antigen-Specific Regulatory T Cells Recognizing the Presented Peptide

In the experiment shown in FIG. 7A, 5×10⁶ Peripheral blood mononuclear cells (PBMC) were taken from two independent healthy donors and co-cultured for 14 days in 2 ml RPMI1640 medium with 5% human serotype AB serum, 5 ng/ml TGF-β1, 20 ng/ml IL-2 (Treg medium) in the presence of 1×10⁶ irradiated JEG-3 cells that had been loaded either with Melan-A or STEAP1 peptides (loading as previously described). At day 3, fresh medium was added. At day 7, medium was exchanged and PBMCs were transferred onto 1×10⁶ freshly irradiated and peptide-loaded JEG-3 cells. Treg Expansion Beads (Miltenyi Biotec, anti CD3/CD28) were used according to the manufacturers protocol as a positive control. The resulting cells were stained for 30 min on ice with antibodies against human CD4 (clone EDU-2, Immunotools) and CD25 (Miltenyi 120-001-311) and with an HLA-A2 STEAP1 dextramer (STEAP1 dex, Immudex Denmark, all dilutions 1:100). The frequency of STEAP1 specific T cells among the CD4⁺ CD25^{high} Treg cells (Shevach et al., 2002, Nat. Rev. Immunol. 2:389) was quantified by flow cytometry. While no STEAP1 specific CD4⁺ CD25^{high} Treg cells were detectable when PBMCs were cultured alone (ctrl) or in presence of a control peptide (melA), a significant population was repeatedly observed when PBMCs were cocultured with JEG-3 cells presenting the cognate antigen, and to a lower extend in the positive control setting (aCD3/28).

In the experiment shown in FIG. 7B, 4×10⁵ DC-10 cells per well were loaded with disulfide trap single chain HLA-G constructs comprising a presented MELAN-A (dtGmelA) or a STEAP1 (dtGsteap) peptide as described for FIG. 6. Then, 4×10⁶ PBLs from the same donor were added, and cells were cultured for 7 days in 12 well plates in 2 ml Treg medium with 1 ml medium being replaced on day 3. On day 7, 4×10⁵ fresh and identically loaded DC-10 were added to each well, medium was again replaced on day 10. At day 14, cells were collected, washed and stained with fluorophore labeled antibodies against CD4 (clone MEM-241), CD8 (clone RPA-T8), and HLA-A2-Melan A peptide dextramers (Immudex). Intracellular staining for IL-10 (clone JES3-9D7) was carried out using an intracellular staining kit (eBiosciences). The number of Melan A specific IL-10⁺ Treg was strongly increased in conditions in which PBLs were cocultured with DC-10 loaded with single chain Melan A HLA-G molecules (dtGmelA) as compared to control molecules (dtGsteap) or untreated PBLs.

### Example 7: Single Chain Peptide MHC Constructs Containing a Human MHC Ib Alpha3 Domain in Combination with DCs Induce Murine Treg Cells Specific for the Presented Peptide (FIG. 8)

Murine DCs (mDCs) were generated by culturing bone marrow cells from wild-type C57BL/6 mice for 7 days in RPMI-1640 complete supplemented with 10% GM-CSF supernatant from an Ag8653 myeloma cell line transfected with the murine GM-CSF gene (detailed protocol: Lutz et al., J Immunol Methods 1999, 223(1):77-92). 4×10⁵ mDCs in 500 μl RPMI complete were combined for 4 h with 500 μl “day 5 CHO supernatants” from mock transfected cells (CHO) or CHO cells transfected with pCDNA3.1 vectors coding for single chain ovalbumin peptide (SIINFEKL), murine H-2Kb alpha 1 and 2 domains and the human HLA-G alpha3 domain (H2Kb, Sequence Example 4 dtH2KbGova) or human HLA-A2 alpha 1 and 2 domains (A2G). The presence of the respective constructs in the supernatant was confirmed by Western Blotting. Preliminary results suggest that an induction is also possible with purified constructs. Here, peptide-loaded MHC constructs were purified using cOmplete His-Tag purification resin (Sigma Aldrich) to bind the constructs, followed by washing with PBS (three times) and Factor Xa Protease digestion (1 U/100 μl, 6 h at 20° C., Qiagen) to release the constructs. Factor Xa can then be removed using factor Xa removal resin (Qiagen, all according to manufacturers protocols)]. Sequences are listed in example 4. mDCs were then washed with PBS.

C57BL/6 RAG^{-/-} OT1 mice express almost exclusively T cell receptors interacting with the ova peptide presented by H-2Kb. 2×10⁶ Splenocytes from these mice were cultured for 14 days in Treg induction medium (RPMI complete, 5 ng/ml IL-2, 5 ng/ml TGF-β1) with (mDC A2G/CHO/H2Kb OT1) or without (OT1 ctrl) 4×10⁵ mDCs loaded as described. Cells were then stained with fluorophore labeled antibodies specific for murine CD3 (clone KT3, Serotec), Foxp3 (3G3, Miltenyi Biotec) and IL10 (JES5-16E3) and quantified by flow cytometry (see Hünig et al., Brain. 2008 September; 131(Pt 9): 2353-65 for mice and protocols). A highly significant increase in antigen-specific Treg was observed in all conditions in which T cells were combined with cognate peptide/MHC alpha 1 & 2 domains and the immunosuppressive alpha 3 domain of an MHC Ib molecule. The moderate induction with purified constructs may be explained by a loss of protein during the purification process.

These experiments imply that peptide presentation on MHC class Ib molecules promotes the expansion of cognate Treg. Such Treg would preferentially be activated via their T cell receptor in tissues in which the antigen is present and should thus enable the targeted tissue-specific suppression of autoimmune reactions provided that a suitable tissue-specific antigen is available. It should be noted that due to the bystander inhibition capacity of antigen-specific Treg the chosen tissue-specific “Treg activation antigen” does not have to be identical to the autoantigen driving the pathological immune response.

### INDUSTRIAL APPLICABILITY

The compositions, polypeptides, nucleic acids, cells, combinations and methods of the invention are industrially applicable. For example, they can be used in the manufacture of, or as, pharmaceutical products.

1. A pharmaceutical composition comprising:
a) a human MHC class Ib molecule, or a polypeptide capable of presenting peptide antigens to T cells, wherein the polypeptide comprises an [alpha] 3 domain of a human MHC class Ib molecule or a derivative of an [alpha] 3 domain of a human MHC class Ib molecule, said derivative being capable of binding to ILT2 or ILT4, and
b) a peptide antigen which is presented by said MHC class Ib molecule or polypeptide according to a).

2. The pharmaceutical composition according to claim 1, wherein the composition comprises the polypeptide capable of presenting peptide antigens according to a), and wherein said polypeptide comprises, preferably in an N- to C-terminal order, an [alpha]1 and an [alpha]2 domain of an MHC class Ia molecule that is followed by said [alpha]3 domain or said derivative.

3. The pharmaceutical composition according to claim 1 or 2, wherein the [alpha]3 domain or derivative comprised by said MHC class Ib molecule or polypeptide is identical to or has at least 80% amino acid sequence identity, preferably at least 90% amino acid sequence identity, with the [alpha]3 domain amino acid sequence of SEQ ID No: 11.

4. The pharmaceutical composition according to claim 3, wherein the [alpha]3 domain or derivative comprised by said MHC class Ib molecule or polypeptide is identical to or has at least 92% amino acid sequence identity with the [alpha]3 domain amino acid sequence of SEQ ID No: 11.

5. The pharmaceutical composition according to claim 3, wherein the [alpha]3 domain or derivative comprised by said MHC class Ib molecule or polypeptide is identical to or has at least 94% amino acid sequence identity with the [alpha]3 domain amino acid sequence of SEQ ID No: 11.

6. The pharmaceutical composition according to claim 3, wherein the [alpha]3 domain or derivative comprised by said MHC class Ib molecule or polypeptide is identical to or has at least 96% amino acid sequence identity with the [alpha]3 domain amino acid sequence of SEQ ID No: 11.

7. The pharmaceutical composition according to claim 3, wherein the [alpha]3 domain or derivative comprised by said MHC class Ib molecule or polypeptide is identical to or has at least 98% amino acid sequence identity with the [alpha]3 domain amino acid sequence of SEQ ID No: 11.

8. The pharmaceutical composition according to claim 3, wherein the [alpha]3 domain or derivative comprised by said MHC class Ib molecule or polypeptide is identical to or has at least 99% amino acid sequence identity with the [alpha]3 domain amino acid sequence of SEQ ID No: 11.

9. The pharmaceutical composition according to claim 3, wherein the [alpha]3 domain or derivative comprised by said MHC class Ib molecule or polypeptide is identical to the [alpha]3 domain amino acid sequence of SEQ ID No: 11.

10. The pharmaceutical composition according to any of the preceding claims, wherein said MHC class Ib molecule according to a) or said polypeptide capable of presenting peptide antigens according to a) is capable of binding to ILT2 or ILT4 with an affinity constant K_{d} of less than 40 μM as measured by surface plasmon resonance spectroscopy.

11. The pharmaceutical composition according to any of the preceding claims, wherein said MHC class Ib molecule according to a) or said polypeptide capable of presenting peptide antigens according to a) is capable of binding to ILT2 or ILT4 with an affinity constant K_{d} of less than 20 μM as measured by surface plasmon resonance spectroscopy.

12. The pharmaceutical composition according to any of the preceding claims, wherein said MHC class Ib molecule according to a) or said polypeptide capable of presenting peptide antigens according to a) is capable of binding to ILT2 or ILT4 with an affinity constant K_{d} of less than 10 μM as measured by surface plasmon resonance spectroscopy.

13. The pharmaceutical composition according to any of the preceding claims, wherein said pharmaceutical composition further comprises a polypeptide domain comprising the amino acid sequence of SEQ ID No: 6, or a sequence at least 90% identical to the amino acid sequence of SEQ ID No: 6, preferably at least 95% identical to the amino acid sequence of SEQ ID No: 6, more preferably at least 98% identical to the amino acid sequence of SEQ ID No: 6, and wherein said polypeptide domain is preferably comprised by the polypeptide capable of presenting peptide antigens according to a).

14. The pharmaceutical composition according to any of the preceding claims, wherein said MHC class Ib molecule according to a) or said polypeptide capable of presenting peptide antigens according to a) further comprises one or more linker sequences, preferably (GGGGS)n linker sequences.

15. The pharmaceutical composition according to any of the preceding claims, wherein said MHC class Ib molecule according to a) or said polypeptide capable of presenting peptide antigens according to a) is a dimer or multimer.

16. The pharmaceutical composition according to any of the preceding claims, wherein the peptide antigen is 7 to 11 amino acids in length, preferably 8-10 amino acids in length.

17. The pharmaceutical composition according to any of claims 1 and 3-16, wherein the composition comprises the MHC class Ib molecule according to a), and wherein the MHC class Ib molecule is HLA-E, HLA-F or HLA-G.

18. The pharmaceutical composition according to claim 17, wherein the MHC class Ib molecule is HLA-G.

19. The pharmaceutical composition according to claim 17 or 18, wherein the MHC class Ib molecule is a human MHC class Ib molecule.

20. The pharmaceutical composition according to any of the preceding claims, wherein the peptide antigen according to b) is covalently bound to the MHC class Ib molecule or polypeptide according to a).

21. The pharmaceutical composition according to claim 20, wherein the peptide antigen according to b) and the MHC class Ib molecule or polypeptide according to a) are covalently bound through a peptide bond and are part of a single polypeptide chain.

22. A recombinant polypeptide capable of presenting a peptide antigen, the recombinant polypeptide comprising, in an N- to C-terminal order,
i) a peptide antigen presented by said recombinant polypeptide;
ii) optionally a first linker sequence;
iii) optionally a sequence of a human polypeptide domain comprising a sequence of a human β2 microglobulin, or an amino acid sequence at least 90% identical to the amino acid sequence of human β2 microglobulin represented by SEQ ID No: 6;
iv) optionally a second linker sequence;
v) optionally an [alpha] 1 domain of an MHC molecule;
vi) optionally an [alpha] 2 domain of an MHC molecule;
vii) an [alpha] 3 domain of an MHC Ib molecule or a derivative of an [alpha] 3 domain of an MHC class Ib molecule, said derivative being capable of binding to ILT2 or ILT4;
viii) optionally a protease cleavage site; and
ix) optionally an affinity tag.

23. The recombinant polypeptide according to claim 22, wherein v) said [alpha]1 domain and vi) said [alpha]2 domain are from an MHC class Ia molecule.

24. The recombinant polypeptide according to claim 22 or 23, wherein the [alpha]3 domain or derivative is identical to or has at least 80% amino acid sequence identity, preferably at least 90% amino acid sequence identity, with the [alpha]3 domain amino acid sequence of SEQ ID No: 11.

25. The recombinant polypeptide according to claim 24, wherein the [alpha]3 domain or derivative is identical to or has at least 92% amino acid sequence identity with the [alpha]3 domain amino acid sequence of SEQ ID No: 11.

26. The recombinant polypeptide according to claim 24, wherein the [alpha]3 domain or derivative is identical to or has at least 94% amino acid sequence identity with the [alpha]3 domain amino acid sequence of SEQ ID No: 11.

27. The recombinant polypeptide according to claim 24, wherein the [alpha]3 domain or derivative is identical to or has at least 96% amino acid sequence identity with the [alpha]3 domain amino acid sequence of SEQ ID No: 11.

28. The recombinant polypeptide according to claim 24, wherein the [alpha]3 domain or derivative is identical to or has at least 98% amino acid sequence identity with the [alpha]3 domain amino acid sequence of SEQ ID No: 11.

29. The recombinant polypeptide according to claim 24, wherein the [alpha]3 domain or derivative is identical to or has at least 99% amino acid sequence identity with the [alpha]3 domain amino acid sequence of SEQ ID No: 11.

30. The recombinant polypeptide according to claim 24, wherein the [alpha]3 domain is identical to the [alpha]3 domain amino acid sequence of SEQ ID No: 11.

31. The recombinant polypeptide according to any of the preceding claims, wherein said polypeptide is capable of binding to ILT2 or ILT4 with an affinity constant K_{d} of less than 40 μM as measured by surface plasmon resonance.

32. The recombinant polypeptide according to any of the preceding claims, wherein said polypeptide is capable of binding to ILT2 or ILT4 with an affinity constant K_{d} of less than 20 μM as measured by surface plasmon resonance.

33. The recombinant polypeptide according to any of the preceding claims, wherein said polypeptide is capable of binding to ILT2 or ILT4 with an affinity constant K_{d} of less than 10 μM as measured by surface plasmon resonance.

34. The recombinant polypeptide according to any of the preceding claims, wherein said polypeptide is a dimer or multimer.

35. The recombinant polypeptide according to any of the preceding claims, wherein said peptide antigen sequence according to i) is 7 to 11 amino acids in length, preferably 8-10 amino acids in length.

36. The recombinant polypeptide according to any of the preceding claims, wherein the polypeptide comprises all of the components i) to vii) but preferably not components viii) to ix).

37. The recombinant polypeptide according to any of claims 22 to 35, wherein the polypeptide comprises all of the components i) to ix).

38. The recombinant polypeptide according to any of the preceding claims, further comprising an N-terminal secretion signal peptide sequence.

39. A pharmaceutical composition according to any of claims 1 to 21, or a recombinant polypeptide according to any of claims 22 to 38, for use in medicine.

40. A pharmaceutical composition according to any of claims 1 to 21, or a recombinant polypeptide according to any of claims 22 to 38, for use in a method for peptide antigen-specific immunomodulation in a subject, said immunomodulation being specific to the peptide antigen that is comprised by the pharmaceutical composition or recombinant polypeptide.

41. The pharmaceutical composition or recombinant polypeptide according to claim 40 for the use according to claim 40, wherein the method for immunomodulation is for inducing immunological tolerance towards the peptide antigen that is comprised by the pharmaceutical composition or recombinant polypeptide.

42. The pharmaceutical composition or recombinant polypeptide according to any of claims 40-41 for the use according to any of claims 40-41, wherein the method for immunomodulation is a method for the suppression of an immune autoimmune disease, for the suppression of an allergy, for the suppression of an immune reaction towards a biotherapeutical drug, for the suppression of an immune reaction towards an embryonic antigen, or for the suppression of an immune reaction towards transplanted cells, tissues or organs.

43. The pharmaceutical composition or recombinant polypeptide according to claim 42 for the use according claim 42, wherein the method for immunomodulation is a method for induction of immune tolerance and wherein the autoimmune disease affects multiple organs, hormone producing organs, nerves, joints, the skin, the gastrointestinal system, the eyes, blood components or blood vessels.

44. The pharmaceutical composition or recombinant polypeptide according to claim 41 for the use according to claim 41, wherein the method is a method for suppression of an immune response in Crohn's disease, ulcerative colitis, systemic lupus erythematosus (SLE), multiple sclerosis, rheumatoid arthritis, psoriasis, scleroderma, neuromyelitis optica or type 1 diabetes.

45. A nucleic acid encoding the polypeptide according to any one of claims 22-38 or the polypeptide or MHC class Ib molecule as defined in any of claims 1-21.

46. The nucleic acid according to claim 45, wherein the nucleic acid is a vector.

47. A pharmaceutical composition comprising the nucleic acid according to claim 45 or 46.

48. A recombinant host cell comprising a nucleic acid molecule or a vector according to claim 45 or 46.

49. A method for producing a polypeptide according to any one of claims 22-38, comprising culturing a recombinant host cell of claim 48 under conditions allowing expression of the nucleic acid molecule, and recovering the polypeptide produced.

50. A combination of
a1) an antigenic protein or peptide antigen, or a nucleic acid encoding said antigenic protein or peptide antigen, or an attenuated organism containing said antigenic protein or peptide antigen or a2) a cell presenting said peptide antigen according to a1);
and
b) an agent capable of blocking the binding between an MHC class Ib molecule and its receptor;
for use in a method of inducing in a human subject an immune response against said antigenic protein or peptide antigen.

51. The combination for use according to claim 50, wherein the agent is capable of binding to said human MHC class Ib molecule and/or its receptors.

52. The combination for use according to any of the preceding claims, wherein the agent is capable of binding to HLA-G.

53. The combination for use according to claims 50-52, wherein the agent is an antibody, preferably a monoclonal antibody, which is capable of binding to HLA-G.

54. The combination for use according to any of the preceding claims, wherein the agent is capable of binding to ILT2 or ILT4.

55. The combination for use according to any of the preceding claims, wherein the agent is an antibody, preferably a monoclonal antibody, which is capable of binding to ILT2 or ILT4.

56. The combination for use according to any of the preceding claims, wherein the agent comprises an Fc domain of an antibody or a fragment thereof.

57. The combination for use according to any of the preceding claims, wherein the agent comprises an [alpha]3 domain of an MHC class Ib molecule.

58. The combination for use according to any of the preceding claims, wherein the agent comprises one or more extracellular domains of ILT2 or ILT4 receptors, preferably at least the two N-terminal extracellular domains of ILT2 or ILT4 receptors, and wherein the agent comprises more preferably a soluble ILT2 or ILT4 receptor.

59. The combination for use according to any of the preceding claims, wherein the agent is to be administered simultaneously with, before, or after administration of said antigenic protein or peptide antigen or said nucleic acid encoding said antigenic protein or peptide antigen or said attenuated organism containing said antigenic protein or peptide antigen.

60. The combination for use according to any of the preceding claims, wherein the combination is a combination of a) an antigenic protein or peptide antigen; and b) an agent capable of blocking the binding between said MHC class Ib molecule and its receptor.

61. The combination for use according to any of claims 50-59, wherein the combination is a combination of a) a nucleic acid encoding an antigenic protein or peptide antigen; and b) an agent capable of blocking the binding between said MHC class Ib molecule and its receptor.

62. The combination for use according to any of claims 50-59, wherein the combination is a combination of a) an attenuated organism containing an antigenic protein or peptide antigen; and b) an agent capable of blocking the binding between said MHC class Ib molecule and its receptor.

63. The combination for use according to claim 62, wherein the attenuated organism containing said antigenic protein or peptide antigen is an attenuated virus.

64. The combination for use according to any of claims 50-62, wherein the antigenic protein or peptide antigen according to a) is a tumor antigen or an antigen that is at least 77% identical to the tumor antigen and is capable of inducing cross-protection against said antigen.

65. The combination for use according to any of the preceding claims, wherein the method is a method for T cell based immunotherapy.

66. The combination for use according to any of claims 50-63 and 65, wherein the antigenic protein or peptide antigen is detectable in pathogenic microorganisms or viruses.

67. The combination for use according to any of the preceding claims, wherein the method is a method for the treatment or prevention of an infectious or malignant disease.

68. The combination for use according to claim 67, wherein the disease is a cancer and wherein the peptide antigen is a tumor antigen.

69. The combination for use according to claim 68, wherein the cancer is selected from the group consisting of melanoma, renal carcinoma, ovarian carcinoma, colorectal cancer, breast cancer, gastric cancer, pancreatic ductal adenocarcinoma, prostate cancer, B and T cell lymphoma and lung cancer.

70. The combination for use according to any of the preceding claims, wherein the combination is present in one pharmaceutical composition.

71. The combination for use according to any of the preceding claims, wherein said immune response against said antigenic protein or peptide antigen is specific to said antigenic protein or peptide antigen.

72. An agent capable of blocking the binding between an MHC class Ib molecule and its receptor as defined in any one of claims 50 to 62, for use in a method for the treatment of a cancer in a human subject, said method including a therapy resulting in a release of cancer antigens from cells of said cancer.

73. The agent for use according to claim 72, wherein said therapy resulting in a release of cancer antigens is chemotherapy or radiotherapy.

74. The pharmaceutical composition or recombinant polypeptide according to claim 41 for the use according to claim 41, wherein the method for inducing immunological tolerance towards the peptide antigen further comprises a peptide drug treatment, and wherein the peptide antigen is 1) identical to the peptide drug or is 2) a fragment of said peptide drug or is 3) a derivative of said fragment of said peptide drug that is capable of inducing immunological tolerance against said peptide drug.

75. The pharmaceutical composition or recombinant polypeptide according to claim 41 for the use according to claim 41, wherein the method for inducing immunological tolerance towards the peptide antigen further comprises a protein drug treatment, and wherein the peptide antigen is 1) a fragment of said protein drug or is 2) a derivative of said fragment of said protein drug that is capable of inducing immunological tolerance against said protein drug.

76. The pharmaceutical composition or recombinant polypeptide according to claim 74 for the use according to claim 74, wherein said peptide drug is to be administered in form of the peptide drug itself.

77. The pharmaceutical composition or recombinant polypeptide according to claim 75 for the use according to claim 75, wherein said protein drug is to be administered in form of the protein drug itself.

78. The pharmaceutical composition or recombinant polypeptide according to claim 74 for the use according to claim 74, wherein said peptide drug is to be administered by means of gene therapy, said gene therapy being a gene therapy with a gene encoding said peptide drug.

79. The pharmaceutical composition or recombinant polypeptide according to claim 75 for the use according to claim 75, wherein said protein drug is to be administered by means of gene therapy, said gene therapy being a gene therapy with a gene encoding said protein drug.
